(19) United States
(12) Patent Application Publication
(10) Pub. No.: US 2026/0144726 A1
(43) Pub. Date: May 28, 2026

(54) POLYOL-FREE COSMETIC COMPOSITION COMPRISING ALPHA HYDROXY ACID ESTER

(71) Applicant: L'OREAL, Paris (FR)

(72) Inventors: Xianzhi ZHOU, Millburn, NJ (US); Jun Liang, Staten Island, NY (US)

(73) Assignee: L'OREAL, Paris (FR)

(21) Appl. No.: 18/962,084

(22) Filed: Nov. 27, 2024

(51) Int. Cl. A61K 8/37; A61Q 5/12

(52) U.S. Cl. CPC A61K 8/375; A61Q 5/12; A61K 2800/262; A61K 2800/31

(57) ABSTRACT

A cosmetic composition comprising alpha hydroxy acid esters is described. The cosmetic composition includes: (a) one or more alpha hydroxy acid esters; (b) one or more fatty alcohols; (c) one or more cationic surfactants; (d) optionally, one or more fatty compounds; wherein the composition is essentially free from water, mono-alcohols having from 2 to 6 carbon atoms, and polyols having from 2 to 8 carbon atoms. The cosmetic composition is typically transparent or translucent, forms a lamellar phase in situ when contact with water, and provides deep conditioning and other benefits to the hair.

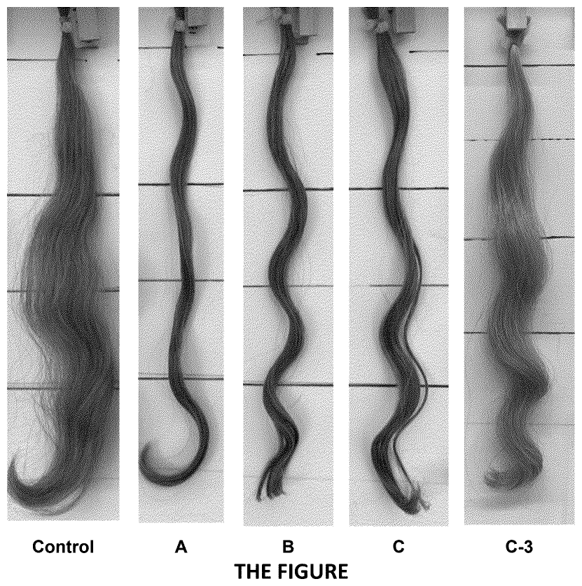

### FIELD OF THE DISCLOSURE

The instant disclosure is drawn to cosmetic compositions containing alpha hydroxy acid esters, and to methods for treating hair, for example, conditioning, managing, and improving the look and feel of hair.

### BACKGROUND

Many individuals suffer from dry and damaged hair. Dryness and damage can occur due to several factors including weather exposure, mechanical treatments (e.g., brushing hair), excessive treatments using chemicals, dying hair, heat styling, etc. In combination, using cleansing products that can be excessively stripping of hair's natural oils can also not only lead to split ends and dull hair, but also exacerbate dry hair. To mitigate the damage, oil treatments, conditioner, hair masks, and chemical treatments are commonly used.

The popularity and usage of oils for dry hair treatments has increased due to their effectiveness and simplicity. Commonly used oils include olive oil, mineral oil, avocado oil, apricot kernel oil, rice bran oil, and coconut oil. However, one problem is that effects are not usually seen after more than several hours (e.g., 8 hours) of treatment and several treatments are usually required, making it time consuming and labor intensive.

Individuals desire natural treatments for hair or damaged hair that is not time consuming and labor intensive to use. A variety of approaches have been developed to condition the hair. A common method of providing conditioning benefit is through using conditioning agents such as cationic surfactants and polymers, high melting point fatty compounds, low melting point oils, silicone compounds, and combinations thereof. Most of these conditioning agents are known to provide various conditioning benefits.

However, there is still a need for providing improved hair manageability, for example, improved hair alignment, reduced unwanted volume (especially reduced frizz), and increased shine. There is also a need to develop hair care products that can impart other benefits at the same time in addition to caring and conditioning benefits, such as styling, volume, shaping, curl definition (for curly or wavy hair), and restyle-ability or reshaping (without the need to reapply the product).

The formulation of environmentally-friendly cosmetic products, which are designed and developed considering environmental issues, is becoming a major goal in an effort to meet global challenges. It is therefore essential to propose more sustainable compositions, preparation processes and ingredients, along with uses of such sustainable compositions, to address these environmental concerns. In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

### BRIEF DESCRIPTION OF THE FIGURES

Implementation of the present technology is described, by way of example only, with reference to the attached FIGURE, wherein:

The FIGURE shows a swatch treated with a cosmetic composition according to the instant disclosure, hair swatches treated with comparative compositions, and a hair swatch serving as a control.

### SUMMARY OF THE DISCLOSURE

The instant disclosure is drawn to an essentially anhydrous cosmetic composition, and to methods for treating hair. For example, the cosmetic composition is useful in methods for conditioning, softening, and reducing the frizz of hair. The composition is unique because it includes a large proportion of one or more alpha hydroxy acid esters, which improves the hair's texture, appearance, and overall health. The composition is typically solubilized, clear (transparent), and free flowing. Upon application to wet or damp hair, or dilution with water, the compositions interacts with the available water and forms a lamellar phase in situ, which enhances deposition of fatty alcohols and/or cationic surfactants to the hair. The cosmetic composition includes:
(a) one or more alpha hydroxy acid esters;
(b) one or more fatty alcohols;
(c) one or more cationic surfactants; and
(d) optionally, one or more fatty compounds;
wherein the composition is essentially free from water, mono-alcohols having from 2 to 6 carbon atoms, and polyols having from 2 to 8 carbon atoms.

The cosmetic composition is in the form of a solubilized, non-emulsified composition that is transparent or translucent, preferably transparent. Upon application to wet or damp hair, or upon dilution with water, the composition forms a lamellar phase in situ to provide deep moisturizing and conditioning benefits to the hair. Available water on the wet or damp hair interacts with the composition prompting formation of a lamellar phase. Additional water can be used to enhance formation of the lamellar phase, for example, when the hair is not very wet.

Nonlimiting examples of alpha hydroxy esters include esters of glycolic acid, lactic acid, malic acid, tartaric acid, citric acid, or combinations thereof. For example, without limitation, the glycolic acid esters include methyl glycolate, ethyl glycolate, n-propyl glycolate, isopropyl glycolate, butyl glycolate, or combinations thereof; the lactic acid esters include methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, or combinations thereof; the malic acid esters include dimethyl malate, diethyl malate, diisopropyl malate, dipropyl malate, dibutyl malate, diisobutyl malate, ditertbutyl malate, dimethyl acetyl malate, diethyl acetyl malate, dipropyl acetyl malate, diisopropyl acetyl malate, dibutyl acetyl malate, diisobutyl acetyl malate, ditertbutyl acetyl malate, or combinations thereof; the tartaric acid esters include dimethyl tartrate, diethyl tartrate, dibutyl tartrate, diisopropyl tartrate, or combinations thereof; and the citric acid esters include trimethyl citrate, triethyl citrate, tri-n-butyl citrate, tributyl citrate, acetyl triethyl citrate, acetyl tributyl citrate, trihexyl citrate, butyl trihexyl citrate, stearyl citrate, ethyl mandelate, ethyl salicylate, methyl salicylate, and ethyl glycolate.

The one or more fatty alcohols may be selected from saturated or unsaturated, linear or branched fatty alcohols having from 10 to 30 carbon atoms, preferably from 12 to 28 carbon atoms, more preferably from 14 to 24 carbon atoms. Nonlimiting examples of fatty alcohols include decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, lauryl alcohol, and a combination thereof. In some instances, the cosmetic compositions preferably include oleyl alcohol, behenyl alcohol, cetearyl alcohol, or combinations thereof.

Nonlimiting examples of cationic surfactants include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and combinations thereof.

In various embodiments, the one or more cationic surfactants include an amidoamine surfactant. Nonlimiting examples of amidoamine surfactants include oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, dilinoleamidopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, stearamidoethyldiethylamine, or a combination thereof.

The cosmetic composition optionally includes one or more fatty compounds. Nonlimiting examples of fatty compounds include propylene glycol dicaprylate/dicaprate, propylene glycol dicaprylate, dicaprylyl carbonate, dicaprylyl ether, dibutyl adipate, C12-15 alkyl benzoate, propylheptyl caprylate, ethylhexyl palmitate, coco-caprylate/caprate, butylene glycol dicaprylate/dicaprate, diisopropyl adipate, ethyl oleate, ethylhexyl hydroxystearate, glycol distearate, glycol stearate, PPG-20 methyl glucose ether distearate, triacetin, sucrose distearate, or combinations thereof.

The composition may be applied to wet or damp hair or diluted with water for use on dry or wet hair. It can serve as a replacement for conditioner when applied after shampooing. Alternatively, it can be used following both shampooing and conditioning, with the option to rinse it out or leave it on, functioning as either a rinse-off or leave-in product. It may also be used as a pre-treatment before shampooing. In some cases, after applying the composition, the hair is rinsed, dried, and styled as desired. In other instances, it is applied as a leave-on treatment, allowing the product to remain on the hair without rinsing before styling. For example, diluted compositions can be applied to hair and left indefinitely to enhance and maintain long-term styling.

### DETAILED DESCRIPTION OF THE DISCLOSURE

This cosmetic composition of the instant case improves the appearance, feel, and manageability of hair, leveraging the benefits of one or more alpha hydroxy acid (AHA) esters. When applied to wet hair, the composition forms a lamellar phase in situ, a layered structure of water and lipids. The lamellar orientation enhances deposition conditioning agents such as fatty alcohols and cationic surfactants, ensuring efficient delivery and distribution along the entirety of the hair fibers. The AHA esters not only contribute to this structured delivery system but also improve hydration by smoothing the hair cuticle and aiding in moisture retention. As a result, the hair feels naturally soft, light, and moisturized without a greasy or weighed-down sensation. Thus, treatment with the composition leaves the hair with increased shine, body, and manageability.

### The Hair Composition Typically Includes:

(a) about 5 to about 99 wt. % of one or more alpha hydroxy acid esters;
(b) about 0.1 to about 10 wt. % of one or more fatty alcohols;
(c) about 0.1 to about 10 wt. % of one or more cationic surfactants; and
(d) optionally, one or more fatty compounds;
   all percentages by weight based on a total weight of the composition.

The composition is preferably free or essentially free from water, and therefore can be characterized as an “anhydrous composition.” The composition is also preferably free or essentially free from mono-alcohols having from 2 to 6 carbon atoms, such as ethanol and isopropanol. Moreover, the composition is also preferably free or essentially free from polyols having from 2 to 8 carbon atoms, such as glycerin and propylene glycol. The cosmetic composition is typically a solubilized, non-emulsified composition that is translucent or transparent.

Upon application to wet or damp hair, or dilution with water, the composition preferably forms a lamellar phase in situ. When contacted with water, the composition will typically transform from a translucent or transparent composition into an opaque composition with a creamy texture. Upon application to wet or damp hair, the composition interacts with the water and becomes luscious.

The term “transparent” with respect to a transparent composition indicates that the composition has a transmittance of at least 80% at a wavelength of 600 nm, for example measured using a Lambda 40 UV-visible spectrometer. The composition may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure.

The term “translucent” with respect to a translucent composition indicates that the composition has a transmittance of at least 50% at a wavelength of 600 nm, for example measured using a Lambda 40 UV-visible spectrometer.

The term “opaque” with respect to an opaque composition indicates the composition is not transparent or translucent, i.e., the composition has a transmittance of less than 50% at a wavelength of 600 nm, for example measured using a Lambda 40 UV-visible spectrometer.

A “lamellar phase” refers generally to packing of polar-headed long chain nonpolar-tail molecules in an environment of bulk polar liquid (i.e., water), as sheets of bilayers separated by bulk liquid.

### (a) Alpha Hydroxy Acid Esters

Alpha Hydroxy Acid (AHA) esters are compounds derived from alpha hydroxy acids (AHAs), which are a class of naturally occurring or synthetic acids characterized by a hydroxyl group (—OH) attached to the carbon adjacent to the carboxylic acid group (—COOH). In their esterified form, AHAs are reacted with alcohols to form esters, which typically have the general structure: R—O—CO—C(—OH)—R′, where R and R′ can be hydrogen or organic groups, in particular, an alkyl group having 2 to 10 carbon atoms, preferably 2 to 6 carbon atoms.

Nonlimiting examples of alpha hydroxy esters include glycolic acid esters, lactic acid esters, malic acid esters, tartaric acid esters, citric acid esters, or combinations thereof.

Nonlimiting examples of glycolic acid esters include those represented by formula: CH₂OH(COOR₅), where R₅ is C₂ to C₆ alkyl group. More specific but nonlimiting examples include methyl glycolate, ethyl glycolate, n-propyl glycolate, isopropyl glycolate, butyl glycolate, or combinations thereof.

Nonlimiting examples of lactic acid esters include those represented by formula: C₂H₄OH(COOR₆), where R₆ is C₂ to C₆ alkyl group. More specific but nonlimiting examples include methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, or combinations thereof.

Nonlimiting examples of malic acid esters include those represented by formula: C₂H₃OH(COOR₇)(COOR₈), where R₇ and R₈ are independently H atom or C₂ to C₆ alkyl group, and one of R₇ and R₈ is an alkyl group. More specific but nonlimiting examples include dimethyl malate, diethyl malate, diisopropyl malate, dipropyl malate, dibutyl malate, diisobutyl malate, ditertbutyl malate, dimethyl acetyl malate, diethyl acetyl malate, dipropyl acetyl malate, diisopropyl acetyl malate, dibutyl acetyl malate, diisobutyl acetyl malate, ditertbutyl acetyl malate, or combinations thereof.

Nonlimiting examples of tartaric acid esters are those represented by formula: C₂H₂(OH)₂(COOR₉)(COOR₁₀), where R₉ and R₁₀ are independently H atom or C₂ to C₆ alkyl group, and one of R₉ and R₁₀ is an alkyl group. More specific but nonlimiting examples include dimethyl tartrate, diethyl tartrate, dibutyl tartrate, diisopropyl tartrate, or combinations thereof.

Nonlimiting examples of citric acid ester include those represented by formula: C₃H₄OH(COOR₁₁)(COOR₁₂)(COOR₁₃), where R₁₁, R₁₂ and R₁₃ are independently H atom or C₂ to C₆ alkyl group, and one of R₁₁, R₁₂ and R₁₃ is an alkyl group. More specific but nonlimiting examples include trimethyl citrate, triethyl citrate, tri-n-butyl citrate, tributyl citrate, acetyl triethyl citrate, acetyl tributyl citrate, trihexyl citrate, butyl trihexyl citrate, stearyl citrate, ethyl mandelate, ethyl salicylate, methyl salicylate, ethyl glycolate, or combinations thereof.

In a preferred embodiment, the one or more alpha hydroxy acid esters are selected from diethyl tartrate, ethyl lactate, triethyl citrate, or combinations thereof.

The total amount of the one or more alpha hydroxy acid esters will vary and may be, for example, from about 5 to about 99 wt. %, based on the total weight of the cosmetic composition. In further embodiments, the cosmetic composition includes about 5 to about 90 wt. %, about 5 to about 80 wt. %, about 5 to about 70 wt. %, about 5 to about 60 wt. %, about 5 to about 50 wt. %, about 5 to about 45 wt. %, about 10 to about 99 wt. %, about 10 to about 90 wt. %, about 10 to about 80 wt. %, about 10 to about 70 wt. %, about 10 to about 60 wt. %, about 10 to about 50 wt. %, about 10 to about 45 wt. %, about 20 to about 99 wt. %, about 20 to about 90 wt. %, about 20 to about 80 wt. %, about 20 to about 70 wt. %, about 20 to about 60 wt. %, about 20 to about 50 wt. %, about 20 to about 45 wt. % of the one or more alpha hydroxy acid esters, based on the total weight of the cosmetic composition.

In further embodiments, the cosmetic composition includes from about 5 to about 40 wt. %, about 5 to about 35 wt. %, about 5 to about 30 wt. %, about 5 to about 25 wt. %, about 10 to about 40 wt. %, about 10 to about 35 wt. %, about 10 to about 30 wt. %, about 10 to about 25 wt. %, about 15 to about 40 wt. %, about 15 to about 35 wt. %, about 15 to about 30 wt. %, about 15 to about 25 wt. %, about 10 to about 40 wt. %, about 10 to about 35 wt. %, about 10 to about 30 wt. %, about 10 to about 25 wt. %, about 15 to about 40 wt. %, about 15 to about 35 wt. %, about 15 to about 30 wt. %, about 15 to about 25 wt. %, or about 17 to about 23 wt. % of the one or more alpha hydroxy acid esters, based on the total weight of the cosmetic composition.

Preferably, the cosmetic composition includes about 5 to about 90 wt. %, more preferably about 10 to about 60 wt. %, and more preferably about 15 to about 50 wt. % of one or more alpha hydroxy acid esters, based on the total weight of the cosmetic composition.

### (b) Fatty Alcohols

The term “fatty alcohol” means an alcohol comprising at least one hydroxyl group (OH), and typically comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In various embodiments, the cosmetic composition includes at least one solid fatty alcohol. Solid fatty alcohols are fatty alcohols that are solid at ambient temperature and at atmospheric pressure (25° C., 780 mmHg), and are insoluble in water, i.e., they have a water solubility of less than 1% by weight, preferably less than 0.5% by weight, at 25° C., 1 atm. The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms. Nonlimiting examples include lauryl alcohol (1-dodecanol); myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricylic alcohol (1-triacontanol), and combinations thereof. In a preferred embodiment, the compositions include at least one solid fatty alcohol selected from myristyl alcohol, cetyl alcohol, stearyl alcohol, behenyl alcohol and combinations thereof such as cetylstearyl or cetearyl alcohol.

In various embodiments, the cosmetic composition includes at least one liquid fatty alcohol, in particular containing C10-C34 and preferably have branched carbon chains and/or have one or more, preferably 1 to 3 double bonds. They are preferably branched and/or unsaturated (C═C double bond) and contain from 12 to 40 carbon atoms. The liquid fatty alcohols may be represented by: R—OH, wherein R denotes a C12-C24 branched or straight alkyl group or an alkenyl group, R being optionally substituted by one or more hydroxy groups. In certain embodiments, the liquid fatty alcohols are selected from branched saturated alcohols. Preferably, R does not contain a hydroxyl group. Nonlimiting examples include oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and combinations thereof. In other embodiments, the compositions are free or essentially free from liquid fatty alcohols, including the liquid fatty alcohols referenced above.

In a preferred embodiment, the one or more fatty alcohols are linear (straight chain) saturated fatty alcohols having from 10 to 30 carbon atoms, preferably from 12 to 28 carbon atoms, more preferably from 14 to 24 carbon atoms. Nonlimiting examples include decyl alcohol, undecyl alcohol, dodecyl alcohol, myristyl alcohol, cetyl alcohol, stearyl alcohol, cetearyl alcohol, behenyl alcohol, myricyl alcohol and combinations thereof.

The total amount of the one or more fatty alcohols in the cosmetic compositions will vary but is typically from about 0.1 to about 10 wt. %, based on the total weight of the cosmetic composition. In further embodiments, the cosmetic composition includes from about 0.1 to about 8 wt. %, about 0.1 to about 6 wt. %, about 0.5 to about 10 wt. %, about 0.5 to about 8 wt. %, about 0.5 to about 6 wt. %, about 1 to about 10 wt. %, about 1 to about 8 wt. %, about 1 to about 6 wt. %, about 2 to about 10 wt. %, about 2 to about 8 wt. %, about 2 to about 6 wt. %, about 3 to about 10 wt. %, about 3 to about 8 wt. %, about 3 to about 6 wt. %, about 4 to about 10 wt. %, about 4 to about 8 wt. %, or about 4 to about 6 wt. %, based on the total weight of the cosmetic composition. Preferably, the cosmetic composition includes from about 1 to about 10 wt. %, more preferably about 2 to about 8 wt. %, and even more preferably about 3 to about 7 wt. % of one or more fatty alcohols, based on the total weight of the cosmetic composition.

### (c) Cationic Surfactants

The term “cationic surfactant” means a surfactant that may be positively charged when it is contained in the compositions according to the instant disclosure. This surfactant may bear one or more positive permanent charges or may contain one or more functional groups that are cationizable. Nonlimiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamidopropyldimethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and a combination thereof.

In a preferred embodiment, the one or more cationic surfactants are selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldimethylamine, behenamidoethyldimethylamine, arachidamidopropyldimethylamine, arachidamidopropyldimethylamine, arachidamidoethyidiethylamine, arachidamidoethyidimethylamine, or combinations thereof. Even more preferably, the one or more cationic surfactants are selected from cetrimonium chloride, behentrimonium chloride, behentrimonium methosulfate, and stearamidopropyldimethylamine.

Cationic surfactants include amidoamine surfactants, which are cationizable. Nonlimiting examples of such amidoamine surfactants include oleamidopropyl dimethylamine, stearamidopropyl dimethylamine, isostearamidopropyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, stearamidoethyldiethylamine, and combinations thereof.

The total amount of the one or more cationic surfactants in the cosmetic compositions will vary but is typically from about 0.1 to about 10 wt. %, based on the total weight of the cosmetic composition. In further embodiments, the cosmetic composition includes from about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, about 1 to about 3 wt. %, about 2 to about 10 wt. %, about 2 to about 8 wt. %, about 2 to about 6 wt. %, about 2 to about 5 wt. %, or about 2 to about 4 wt. % of the one or more cationic surfactants, based on a total weight of the cosmetic composition. Preferably, the cosmetic composition includes from about 0.5 to about 8 wt. %, more preferably about 1 to about 6 wt. %, and even more preferably, about 2 to about 5 wt. % of one or more cationic surfactants, based on the total weight of the cosmetic composition.

### Weight Ratio of (b) to (c)

The weight ratio of the one or more fatty alcohols of (b) to the one or more cationic surfactants of (c) will vary but it typically from about 8:1 to about 1:5 ((b): (c)). In further embodiments, the weight ratio of the one or more fatty alcohols of (b) to the one or more cationic surfactants of (c) is from about 6:1 to about 1:4, about 5:1 to about 1:3, about 4:1 to about 1:1, about 3:1 to about 1:1, about 2:1 to about 1:1 ((b): (c)). Preferably, the weight ratio of the one or more fatty alcohols of (b) to the one or more cationic surfactants of (c) is from about 4:1 to about 1:2, more preferably about 3:1 to about 1:1, and even more preferably about 2:1 to about 1:1 ((b): (c)).

### (d) Fatty Compounds

For purposes of the instant disclosure the term “fatty compounds” are understood to be non-silicone fatty compounds. The term “fatty compound” is interchangeable with the term “fatty substance,” and means an organic compound without silicone that is insoluble in water at ordinary temperature (25° C.) and at atmospheric pressure (760 mmHg), i.e., which has a solubility of less than 5%, preferably less than 1% and even more preferentially less than 0.1%. They have in their structure a hydrocarbon-based chain containing at least 6 carbon atoms. Also, for purposes of the instant disclosure, fatty alcohols are not included as a “fatty compound” because they are separately identified as a component of the cosmetic composition. Nonlimiting examples of fatty compounds include fatty acids, triglycerides, phospholipids, steroids, waxes, oils, glycerides, sphingolipids, eicosanoids, alkylglycerols, polyketides, fatty acid amides, and ether lipids. Fatty compounds may be classified as fatty esters, fatty ethers, propylene glycol fatty acid esters, fatty carbonate esters, oils, and waxes.

i. Fatty Esters

Nonlimiting examples of fatty esters include fatty esters from a C₆-C₃₂ fatty acid and/or a C₆-C₃₂ fatty alcohol. These esters may be esters of saturated or unsaturated, linear or branched C₁-C₂₆ aliphatic mono or polyacids and of saturated or unsaturated, linear or branched C₁-C₂₆ aliphatic mono or polyalcohols, the total number of carbon atoms in the esters being greater than or equal to 10. In some cases, for the esters of monoalcohols, at least one of the alcohol or the acid from which the esters of the invention result is branched. Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate, and isostearyl neopentanoate. In various embodiments, the compositions of the instant disclosure may include cetyl esters. Cetyl esters are a mixture of the following esters of saturated fatty acids and fatty alcohols: cetyl palmitate, cetyl stearate, myristyl myristate, myristyl stearate, cetyl myristate, and stearyl stearate.

Mention is made of esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy non-sugar alcohols may also be used. Mention may be made in particular of diethyl sebacate, diisopropyl sebacate, diisopropyl adipate, di-n-propyl adipate, triisopropyl citrate, glyceryl trilactate, glyceryl trioctanoate, neopentyl glycol diheptanoate, and diethylene glycol diisononanoate.

Nonlimiting examples of liquid esters (ester oils) or liquid fatty esters that may be mentioned include, for example, sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, coconut oil, camelina oil, tamanu oil, babassu oil and pracaxi oil, jojoba oil, and shea butter oil, and caprylic/capric triglyceride.

Nonlimiting examples of solid fatty esters include solid esters obtained from C₉-C₂₆ fatty acids and from C₉-C₂₆ fatty alcohols. Among these esters, mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate or stearyl myristate, and hexyl stearate.

In various embodiments, the one or more fatty compound may be selected from cetyl esters, purcellin oil (cetearyl octanoate), isopropyl myristate, isopropyl palmitate, C12-C15 alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, pentaerythritol esters, diisostearyl malate, neopentyl glycol dioctanoate, dibutyl sebacate, di-C12-13 alkyl malate, distearyl dimer dilinoleate, dicetyl adipate, diisooctyl adipate, diisononyl adipate, diisostearyl dimer dilinoleate, diisostearyl fumarate, or combinations thereof.

The esters of fatty acids and/or of fatty alcohols, are esters of saturated or unsaturated, linear or branched C1-C26 aliphatic mono- or polyacids and of saturated or unsaturated, linear or branched C1-C26 aliphatic mono- or polyalcohols, the total carbon number of the esters more particularly being greater than or equal to 10. Among the monoesters, mention may be made of dehydroabietyl behenate; octyldodecyl behenate; isocetyl behenate; cetyl lactate; C12-C15 alkyl lactate; isostearyl lactate; lauryl lactate; linoleyl lactate; oleyl lactate; (iso) stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate. Still within the context of this variant, esters of C4-C22 dicarboxylic or tricarboxylic acids and of C1-C22 alcohols and esters of mono-, di- or tricarboxylic acids and of C2-C26 di-, tri-, tetra- or pentahydroxy alcohols may also be used.

In various embodiments, the one or more fatty compounds include monoester fatty compounds. Nonlimiting examples include ethyl oleate, isopropyl myristate, isopropyl palmitate, isopropyl stearate, methyl laurate, methyl stearate, butyl oleate, cetyl stearate, stearyl alcohol acetate, butyl stearate, octyl stearate, isopropyl caprylate, isopropyl laurate, isopropyl linoleate, propylene glycol monolaurate, propylene glycol monopalmitate, glycerol monooleate, glycerol monostearate, methyl palmitate, butyl palmitate, cetyl palmitate, decyl oleate, octyl oleate, ethyl stearate, ethyl laurate, ethyl palmitate, ethyl caprylate, ethyl decanoate, butyl caprate, octyl decanoate, methyl decanoate, butyl myristate, hexyl myristate, isocetyl stearate, isostearyl alcohol acetate, isopropyl caprate, neopentyl glycol monolaurate, neopentyl glycol monopalmitate, diethylhexyl laurate, propylene glycol monostearate, butyl laurate, stearyl alcohol caprate, glyceryl monostearate, glyceryl monopalmitate, ethylcaprylate, butyl stearate, diisopropyl oleate, diethyl hexanoate, methyl hexanoate, ethyl hexanoate, diisopropyl linoleate, isopropyl stearoyl stearate, butylene glycol monolaurate, butylene glycol monostearate, isopropyl myristate, isopropyl arachidonate, decyl palmitate, and cetyl caprate.

In various embodiments, the one or more fatty compounds include diester fatty compounds. Nonlimiting examples include diethyl succinate, dibutyl succinate, diethylhexyl succinate, diisopropyl sebacate, diethyl sebacate, dibutyl sebacate, diethylhexyl malate, diethylhexyl maleate, dicapryl adipate, dicaprylyl maleate, neopentyl glycol dicaprate, propylene glycol dicaprylate/dicaprate, neopentyl glycol diisostearate, neopentyl glycol diethylhexanoate, neopentyl glycol diheptanoate, diisopropyl adipate, diisopropyl dilaurate, dipropylene glycol dicaprylate, diethylhexyl adipate, diethylhexyl caprate, diisobutyl caprate, neopentyl glycol dipalmitate, ethylhexyl caprate, ethylhexyl caprylate, isopropyl caprate, butylene glycol dicaprylate, butylene glycol dicaprate, dipropylene glycol dicaprate, methyl diethylsuccinate, propylene glycol dodecylbenzoate, methyl ethylcaprylate, propylene glycol laurate, octyl dodecylbenzoate, diethoxyethyl hexanoate, ethylhexyl hexanoate, isononyl isononanoate, diethylene glycol dipropionate, propylene glycol dicaprylate, dimethyl adipate, diethyl isononanoate, diisopropyl dilinoleate, dimethyl sebacate, ethyl hexanoate, diisopropyl octanoate, isopropyl dicaprylate, propyl palmitate, ethyl stearate, diethyl decanoate, decyl adipate, diisopropyl stearate, methyl palmitate, methyl laurate, butyl laurate, diethyl myristate, hexyl laurate, ethyl esters of fatty acids, isopropyl myristate, diacetylated propylene glycol, butyl oleate, isopropyl stearate, butyl myristate, diisobutyl adipate, ethyl oleate, cetyl stearate, polyol esters of fatty acids, ethyl decanoate, ethyl hexyl stearate, diethyl stearate, butyl caprylate, methyl ester of lauric acid, hexyl caprate, dicaprylyl carbonate, and ethyl caprylate.

In various embodiments, the one or more fatty compounds include tri-ester fatty compounds. Nonlimiting examples include glycerol trioleate, glycerol tricaprylate, glycerol tricaprate, glycerol tristearate, glycerol trilaurate, glycerol triacetate, triethyl citrate, triisostearyl citrate, trimethylolpropane trioleate, trimethylolpropane tricaprylate, trimethylolpropane tricitrate, trimethylolpropane trioctanoate, trimethylolpropane tripalmitate, triacetin, triglyceride of caprylic acid, triglyceride of capric acid, triglyceride of stearic acid, triglyceride of lauric acid, triglyceride of oleic acid, tri-n-butyl citrate, tri-n-octyl citrate, trimethylolpropane triisostearate, triacetylglycerol, glyceryl trioleate, glyceryl trilinoleate, trimethylolpropane trisunflower oil, glyceryl tricaprylate, glyceryl tricaprate, glyceryl tristearate, glyceryl triarachidonate, glyceryl trilaurate, triglyceride of linoleic acid, triethylhexanoin, and triheptanoin.

ii. Fatty Ethers

Nonlimiting examples of fatty ethers include olyoxyethylene cetyl/stearyl ether, polyoxyethylene cholesterol ether, polyoxyethylene laurate or dilaurate, polyoxyethylene stearate or distearate, polyoxyethylene lauryl or stearyl ether, dicaprylyl ether, dicetyl ether distearyl ether, dodecyl ether, dilauryl ether, dimyristyl ether, diisononyl ether, or a combination thereof. Non-limiting examples of suitable polyoxyethylene fatty ethers include, but are not limited to, polyoxyethylene cetyl/stearyl ether, polyoxyethylene cholesterol ether, polyoxyethylene laurate or dilaurate, polyoxyethylene stearate or distearate, polyoxyethylene lauryl or stearyl ether, and combinations thereof, wherein the polyoxyethylene head group ranges from about 2 to about 100 groups. In certain embodiments, the polyoxyethylene fatty ethers include polyoxyethylene stearyl ether, polyoxyethylene myristyl ether, polyoxyethylene lauryl ether having from about 3 to about 10 oxyethylene units and combinations thereof. In yet another embodiment, at least one of the emollients is a fatty ether selected from stearyl ether, dicaprylyl ether, dicetyl ether distearyl ether, dodecyl ether, dilauryl ether, dimyristyl ether, diisononyl ether, or a combination thereof.

iii. Propylene Glycol Fatty Acid Esters

Nonlimiting examples of propylene glycol fatty acid esters include propylene glycol esters of medium chain fatty acids (fatty acids having from 6 to 12 carbon atoms), such as propylene glycol dicaprylate/dicaprate, propylene glycol dipelargonate, and propylene glycol dilaurate. A preferred propylene glycol fatty acid ester is propylene glycol dicaprylate/dicaprate. The term “propylene glycol dicaprylate/dicaprate” is understood by those in the art to refer to a combination containing propylene glycol dicaprylate, propylene glycol dicaprylate-caprate, and propylene glycol dicaprate, which may vary in the ratio of these components. An example of a commercially available form of propylene glycol dicaprylate/dicaprate is “CAPTEX® 200,” available from the Abitec Corp. (Columbus, OH, USA).

iv. Fatty Carbonate Esters

Nonlimiting examples of fatty carbonate esters include dialkyl carbonates of formula: R₁O(C═O)OR₂, wherein R₁ and R₂ are independently linear or branched, saturated or unsaturated alkyl chains having 1 to 30 carbon atoms, or having 2 to 28 carbon atoms, or having 4 to 25 carbon atoms, or having 6 to 22 carbon atoms, preferably one or more fatty carbonates selected from C14-15 dialkyl carbonate, dicaprylyl carbonate, diethyl carbonate, dihexyl carbonate, diethylhexyl carbonate, dimethoxyphenyl phenyloxyethyl ethylcarbonate, dimethyl carbonate, dipropyl carbonate, dipropylheptyl carbonate, dioctyl carbonate, and a combination thereof.

iv. Oils

The term “oil” means any fatty substance that is in liquid form at room temperature (20-25° C.) and at atmospheric pressure. The one or more oils can be hydrocarbon-based oils, fluoro oils, non-fluoro oils, or combinations thereof. The term “hydrocarbon-based oil” is interchangeable with the term “hydrocarbon oil.” The one or more oils can be “volatile oils” or “non-volatile oils.” For the purposes of the instant disclosure, the term “volatile oil” means an oil (or non-aqueous medium) capable of evaporating on contact with the skin in less than one hour, at room temperature and at atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40,000 Pa (10-3 to 300 mmHg), preferably ranging from 1.3 Pa to 13,000 Pa (0.01 to 100 mmHg), and preferentially ranging from 1.3 Pa to 1300 Pa (0.01 to 10 mmHg).

Suitable oils include, but are not limited to, natural oils, such as coconut oil; hydrocarbons, such as mineral oil and hydrogenated polyisobutene; fatty alcohols, such as octyldodecanol; esters, such as C₁₂-C₁₅ alkyl benzoate; diesters, such as propylene dipelargonate; and triesters, such as glyceryl trioctanoate. Non-limiting examples of oils that may, optionally, be included in the hair treatment compositions include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names “Miglyol® 810,” “Miglyol® 812,” and “Miglyol® 818” by the company Dynamit Nobel, jojoba oil and shea butter oil.

The fluoro oils may be chosen from perfluoromethylcyclopentane and perfluoro-1,3-dimethylcyclohexane, sold under the names “Flutec® PC1” and “Flutec® PC3” by the company, BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company, 3M, or bromoperfluorooctyl sold under the name “For Alkyl®” by the company, Atochem; nonafluoromethoxybutane and nonafluoroethoxyisobutane; perfluoromorpholine derivatives such as 4-trifluoromethyl perfluoromorpholine sold under the name “PF 5052®” by the company, 3M.

### V. Waxes

Waxes are solids at room temperature and typically have a melting point greater than 30° C. or greater than 30° C. to about 100° C. Natural waxes include animal, vegetable/plant, mineral, or petroleum derived waxes. They are typically esters of fatty acids and long chain alcohols. Wax esters are derived from a variety of carboxylic acids and a variety of fatty alcohols.

Nonlimiting examples of waxes include aliphatic esters, such as cetyl esters, stearyl esters, acacia, beeswax, ceresin, flower wax, citrus wax, carnauba wax, jojoba wax, Japanese wax, polyethylene, microcrystalline, rice bran, lanolin wax, mink, montan, bayberry, ouricury, ozokerite, palm kernel wax, paraffin, avocado wax, apple wax, shellac wax, salvia wax, candelilla wax, their polyalkylene glycol derivatives (e.g., PEG 6-20 beeswax or PEG-12 carnauba wax), and mixtures of any of the aforementioned waxes.

Additional nonlimiting examples of waxes include beeswax, hydrogenated alkyl olive esters (commercially available under the tradename “Phytowax® Olive”), carnauba wax, candelilla wax, ouricury wax, Japan wax, cork fibre wax or sugar cane wax, rice wax, montan wax, paraffin wax, lignite wax or microcrystalline wax, ceresin or ozokerite, palm kernel glycerides/hydrogenated palm glycerides and hydrogenated oils such as hydrogenated castor oil or jojoba oil, sugarcane, retamo, bayberry, rice bran, soy, castor, esparto, hydroxyoctacosyl hydroxystearate, Chinese wax, cetyl palmitate, lanolin, shellac, and spermaceti; synthetic waxes such as those of the hydrocarbon type and polyethylene waxes obtained from the polymerization or copolymerization of ethylene, and Fischer-Tropsch® waxes, or else esters of fatty acids, such as octacosenyl stearate, glycerides which are solid at temperatures of above 30° C.

In various embodiments, it can be useful to include one or more polar fatty compounds. A polar fatty compound refers to a molecule that exhibits both fatty characteristics (due to long-chain aliphatic or acyl groups) and polar properties (due to the presence of functional groups that can interact with water or other polar molecules, such as hydroxyl, ester, or ether groups). These compounds are often amphiphilic, meaning they possess both hydrophilic (water-attracting) and lipophilic (fat-attracting) regions, making them versatile in applications such as emulsification, solubilization, and conditioning. An example is propylene glycol dicaprylate/dicaprate, which is an ester formed from the combination of propylene glycol (a polar diol) with caprylic and capric fatty acids.

In a preferred embodiment, the one or more fatty compounds are selected from propylene glycol dicaprylate, propylene glycol dicaprylate/dicaprate, dicaprylyl carbonate, dicaprylyl ether caprylic/capric triglyceride, butylene glycol dicaprylate/dicaprate, butylene glycol dicaprylate, ethylhexyl dicaprylate, ethylhexyl dicaprylate/dicaprate, coco-caprylate/caprate, cetearyl alcohol caprylate, isopropyl myristate, isopropyl caprylate, isocetyl stearate, octyl palmitate, diethylhexyl carbonate, diisopropyl adipate, diethylhexyl dodecyl malate, neopentyl glycol dicaprate, neopentyl glycol dicaprylate, isopropyl caprate, octyldodecyl stearoyl stearate, glyceryl caprylate, octyl stearate, decyl oleate, hexyl laurate, and methyl caprylate.

The total amount of the one or more fatty compounds, if present, will vary but is typically from about 1 to about 95 wt. %, based on the total weight of the cosmetic composition. In further embodiments, the cosmetic composition includes from about 1 to about 90 wt. %, about 1 to about 80 wt. %, about 1 to about 75 wt. %, about 5 to about 95 wt. %, about 5 to about 90 wt. %, about 5 to about 80 wt. %, about 5 to about 75 wt. %, about 10 to about 95 wt. %, about 10 to about 90 wt. %, about 10 to about 80 wt. %, about 10 to about 75 wt. %, about 20 to about 95 wt. %, about 20 to about 90 wt. %, about 20 to about 80 wt. %, about 20 to about 75 wt. %, about 30 to about 95 wt. %, about 30 to about 90 wt. %, about 30 to about 80 wt. %, about 30 to about 75 wt. %, about 40 to about 95 wt. %, about 40 to about 90 wt. %, about 40 to about 80 wt. %, about 40 to about 75 wt. %, about 50 to about 95 wt. %, about 50 to about 90 wt. %, about 50 to about 80 wt. %, about 50 to about 75 wt. %, about 60 to about 95 wt. %, about 60 to about 90 wt. %, about 60 to about 80 wt. %, about 60 to about 75 wt. %, or about 65 to about 75 wt. %, based on the total weight of the cosmetic composition, based on the total weight of the cosmetic composition. Preferably, the cosmetic composition includes from about 10 to about 95 wt. %, more preferably about 40 to about 90 wt. %, and even more preferably about 50 to about 80 wt. % of one or more fatty compounds, based on the total weight of the cosmetic composition.

### (e) Miscellaneous Ingredients

The cosmetic composition optionally includes (or optionally excludes) one or more miscellaneous ingredients. Miscellaneous ingredients are ingredients that are compatible with the cosmetic compositions and do not disrupt or materially affect the basic and novel properties of the compositions. Nonlimiting examples of miscellaneous ingredients include preservatives, fragrances, pH adjusters, salts, chelating agents, buffers, antioxidants, flavonoids, vitamins, botanical extracts, UV filtering agents, proteins, protein hydrolysates and/or isolates, fillers (e.g., organic and/or inorganic fillers such as talc, calcium carbonate, silica, etc.) composition colorants, etc.

In various embodiments, the composition includes one or more miscellaneous ingredients selected from preservatives, fragrances, pH adjusters, salts, chelating agents, buffers, composition colorants, fillers (such as talc, calcium carbonate, silica, including hydrated silica), vitamins, botanical extracts, and a combination thereof. For example, the compositions may include fragrances, vitamins, preservatives, tocopherol, or combinations thereof. In the context of the instant disclosure, a “composition colorant” is a compound that colors the composition but does not have an appreciable coloring effect on hair. A composition colorant is included to provide a color to the composition for aesthetic appeal but is not intended to impart coloring properties to hair. As an example, hair styling gels can be found in a variety of different colors (e.g., light blue, light pink, etc.), yet application of the styling gel to hair does not visibly change the color of the hair.

The total amount of the one or more miscellaneous ingredients, if present, will vary but is typically from about 0.01 to about 10 wt. %, based on the total weight of the cosmetic composition. In further embodiments, the cosmetic composition includes from about 0.01 to about 8 wt. %, about 0.01 to about 5 wt. %, about 0.1 to about 10 wt. %, about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, or about 1 to about 5 wt. % of one or more miscellaneous ingredients, based on the total weight of the cosmetic composition. Preferably, the cosmetic composition includes from about 0.1 to about 10 wt. %, more preferably about 0.5 to about 8 wt. %, and even more preferably about 1 to about 5 wt. % of one or more miscellaneous ingredients, based on the total weight of the cosmetic composition.

### Water, Mono-Alcohols, and Polyols

The cosmetic composition is free or essentially free from water, is free or essentially free from mono-alcohols having from 2 to 6 carbon atoms, and is free or essentially free from polyols having from 2 to 8 carbon atoms. Mono-alcohols are a subset of alcohols and are characterized by having only one hydroxyl functional group, distinguishing them from polyols (with multiple-OH groups).

The amount of water, if present, is typically less than about 5 wt. %, based on the total weight of the cosmetic composition. Preferably, the amount of water is less an about 4 wt. %, less than about 3 wt. %, less than about 2 wt. %, less than about 1 wt. %, less than about 0.5 wt. %, or less than about 0.1 wt. %, based on the total weight of the cosmetic composition.

Nonlimiting examples of mono-alcohols having from 2 to 8 carbon atoms include ethanol, propanol, isopropanol, butanol, butan-2-ol, 2-methylpropan-1-ol (isobutanol), pentan-1-ol, pentan-2-ol, 2-methylbutan-1-ol, 3-methylbutan-1-ol (Isoamyl alcohol), hexan-1-ol, hexan-2-ol, heptan-1-ol, and octan-1-ol. The amount of mono-alcohols having from 2 to 8 carbon atoms, if present, is typically less than about 5 wt. %, based on the total weight of the cosmetic composition. Preferably, the amount of mono-alcohols having from 2 to 8 carbon atoms is less an about 4 wt. %, less than about 3 wt. %, less than about 2 wt. %, less than about 1 wt. %, less than about 0.5 wt. %, or less than about 0.1 wt. %, based on the total weight of the cosmetic composition.

Nonlimiting examples of polyols having from 2 to 8 carbon atoms include glycerin, ethylene glycol, propylene glycol, butylene glycol, pentylene glycol, hexylene glycol, sorbitol, mannitol, erythritol, xylitol, maltitol, isomalt, caprylyl glycol, dipropylene glycol, diglycerin, polyglycerin-3, polyglycerin-6, polyglycerin-10, neopentyl glycol, trimethylolpropane, 1,2-octanediol. The amount of polyols having from 2 to 8 carbon atoms, if present, is typically less than about 5 wt. %, based on the total weight of the cosmetic composition. Preferably, the polyols having from 2 to 8 carbon atoms are in an amount less an about 4 wt. %, less than about 3 wt. %, less than about 2 wt. %, less than about 1 wt. %, less than about 0.5 wt. %, or less than about 0.1 wt. %, based on the total weight of the cosmetic composition.

In a preferred embodiment, the composition includes less than 5 wt. % of a combination of water, mono-alcohols having from 2 to 6 carbon atoms, and polyols having from 2 to 8 carbon atoms, based on the total weight of the cosmetic composition. The combined amounts of the combined amount of the water, the mono-alcohols having from 2 to 6 carbon atoms, and the polyols having from 2 to 8 carbon atoms is preferably less than 3 wt. %, and more preferably less than 1 wt. %, based on the total weight of the cosmetic composition.

### Additional Embodiments

In various embodiments, the cosmetic compositions optionally includes or excludes one or more polyethylene glycols (PEG), anionic surfactants, nonionic surfactants, anionic polymers, silicones, or combinations thereof. For example, the cosmetic composition may optionally include one or more polyethylene glycols, for example, those having at least 2, 3, 5, 10, or 100 repeating units. In other embodiments, the cosmetic composition is preferably free or essentially free from polyethylene glycols including those having at least 2, 3, 5, 10, or 100 repeating units. The amount of polyethylene glycols, if present is typically less than about 5 wt. %, based on the total weight of the cosmetic composition. More preferably, the polyethylene glycols are in an amount less an about 4 wt. %, less than about 3 wt. %, less than about 2 wt. %, less than about 1 wt. %, less than about 0.5 wt. %, or less than about 0.1 wt. %, based on the total weight of the cosmetic composition.

In various embodiments, the cosmetic composition optionally includes or excludes one or more anionic surfactants. Nonlimiting examples of anionic surfactants include sulfate surfactants, isethionate surfactants, sarcosinate surfactants, sulfonate surfactants, taurate surfactants, etc. The amount of anionic surfactants, if present is typically less than about 5 wt. %, based on the total weight of the cosmetic composition. Preferably, the anionic surfactants are in an amount less an about 4 wt. %, less than about 3 wt. %, less than about 2 wt. %, less than about 1 wt. %, less than about 0.5 wt. %, or less than about 0.1 wt. %, based on the total weight of the cosmetic composition.

In various embodiments, the cosmetic composition optionally includes or excludes one or more nonionic surfactants. A nonionic surfactant is a type of surfactant that does not carry an electrical charge in its hydrophilic (water-attracting) head group. These surfactants are characterized by their ability to stabilize formulations, enhance emulsification, and improve the solubility of ingredients without reacting with ions in the solution. The hydrophilic part of nonionic surfactants typically consists of functional groups like hydroxyl (—OH) or ethylene oxide chains. The amount of nonionic surfactants, if present is typically less than about 5 wt. %, based on the total weight of the cosmetic composition. Preferably, the nonionic surfactants are in an amount less an about 4 wt. %, less than about 3 wt. %, less than about 2 wt. %, less than about 1 wt. %, less than about 0.5 wt. %, or less than about 0.1 wt. %, based on the total weight of the cosmetic composition.

In various embodiments, the cosmetic composition optionally includes or excludes one or more anionic polymers. The term “anionic polymer” is understood as meaning those polymers which carry in a protic solvent under standard conditions at least one structural unit having permanently anionic groups, the anionic groups having to be compensated by counterions while maintaining electroneutrality. As contemplated herein, anionic groups are, for example, carboxylate, sulfate, or sulfonate groups. Nonlimiting examples of anionic polymers include anionic polyurethanes, sodium polynaphthalene sulfonate, sodium lignosulfonate, sodium carboxymethyl cellulose, sodium salt of hydrophobically modified maleic anhydride copolymer, sodium polyacrylate, sodium polymethacrylate, ammonium polyacrylate, ammonium polymethacrylate, sodium salt of polymethacrylic acid, polystyrene sulfonate salts, carrageenan salts, dextran sulfate salts, polyacrylic acid salts, poly(methacrylic acid) salts, alginic acid salts, carboxymethylcellulose salts, polystyrene sulfonate/polystyrene copolymer salts, polystyrene sulfonate/maleic acid copolymers salt, copolymers thereof and combinations thereof. The amount of anionic polymers, if present is typically less than about 5 wt. %, based on the total weight of the cosmetic composition. Preferably, the anionic polymers are in an amount less an about 4 wt. %, less than about 3 wt. %, less than about 2 wt. %, less than about 1 wt. %, less than about 0.5 wt. %, or less than about 0.1 wt. %, based on the total weight of the cosmetic composition.

In various embodiments, the cosmetic composition optionally includes or excludes one or more silicones. Nonlimiting examples of silicones include polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy) diphenylsiloxanes, diphenyl dimethicones, diphenyl(methyldiphenyl)trisiloxanes or 2-phenylethyl)trimethylsiloxysilicates. The amount of silicones, if present is typically less than about 5 wt. %, based on the total weight of the cosmetic composition. Preferably, the silicones are in an amount less an about 4 wt. %, less than about 3 wt. %, less than about 2 wt. %, less than about 1 wt. %, less than about 0.5 wt. %, or less than about 0.1 wt. %, based on the total weight of the cosmetic composition.

### Methods

The cosmetic compositions are useful for treating the hair, for example, for conditioning, managing, and improving the look and feel of hair. The compositions can be applied to wet or damp hair or diluted with water and then applied to hair. Upon application to the hair, the cosmetic composition may be massaged into the hair, for example, with the hands, and/or spread throughout the hair with a comb or brush. This results in a smoothing and softening of the hair, which reduces frizz, dryness, and unwanted volume. The compositions can remain on the hair indefinitely or can be rinsed from the hair prior to drying and styling of the hair. Preferably, the cosmetic compositions are rinsed from the hair prior to drying and styling the hair after being applied and massaged into the hair.

The methods of treating hair according to the instant disclosure include methods according to various routines. For instance, the compositions may be mixed with a shampoo (or conditioner) prior to application to the hair. Alternatively, the composition may be layered on top of (or lathered into) hair upon which the shampoo (or conditioner) is already applied. Furthermore, the composition may be applied separate from the shampoo (or conditioner), i.e., applied to the hair after the shampoo (or conditioner) has been rinsed from the hair. In some instances, it is preferable to treat the hair with a composition of the instant disclosure prior to shampooing the hair, e.g., apply the composition to wet or damp hair prior to application of a shampoo to the hair. The hair may additionally (optionally) be treated with a conditioner after shampooing. In other embodiments, it is preferable to treat the hair with the composition after shampooing and optionally conditioning the hair, while the hair is damp or wet.

### Kits

The cosmetic compositions may be incorporated into a kit. For example, the kits may include at least one cosmetic composition according to the instant disclosure and one or more additional compositions, for example, a shampoo, a conditioner, etc. The various compositions are separately contained in the kits. In some instances, the kits include one or more compositions according to the instant disclosure and a shampoo, both of which are separately contained. The kits may also include one or more compositions according to the instant disclosure, a shampoo, and a conditioner, all of which are separately contained. Instructions, mixing components, brushes, gloves, measuring tools, etc., may optionally be included in the kits.

The cosmetic compositions may be packaged in a variety of different containers, such as, for example, a ready-to-use container. Nonlimiting examples of useful packaging include tubes, jars, caps, unit dose packages, and bottles, including squeezable tubes and bottles. In various embodiments, the cosmetic composition of the instant disclosure are packaged as a spray product or included in a spray bottle, which allows a user to apply the compositions to the hair by spraying the composition onto the hair. The spray product or spray bottle may be a pump spray bottle that is manually actuated or the spray bottle can be pressurized such that the composition is dispensed from a pressurized aerosol container. A propellant may be used to force the composition out of the container. Suitable propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed.

In various embodiments, the cosmetic composition may optionally be free or essentially free from dicaprylyl ether. In other embodiments, the cosmetic composition may optionally be free or essentially free from dicaprylyl carbonate. In yet other embodiments, the cosmetic composition may be free or essentially free from isopropylidene glycerol. In another embodiment, the cosmetic composition may be free or essentially free from dicaprylyl ether, dicaprylyl carbonate, and isopropylidene glycerol.

Methods of treating hair typically include applying a cosmetic composition to wet or damp hair, allowing the composition to remain on the hair for a sufficient amount of time, and rinsing the compositions from the hair. The composition may be applied to the hair in a sequence with other compositions. For example, the composition may be applied to the hair before shampooing the hair, after shampooing the hair, before conditioning the hair, and/or after conditioning the hair. The composition, however, is not required to be used in a sequence. It can be used independently from a shampoo and condition, as needed. The composition may be allowed to remain on the hair for a minimum amount of time before being rinsed from the hair, but it is not necessary to allow the composition to remain on the hair. Conveniently, the compositions can be applied and allowed to remain on the hair for a period of time that is typical for regular shampooing and/or conditioning. For example, the composition (whether combined with another hair-treatment composition such as a shampoo or conditioner) may be applied to the hair and allowed to remain on the hair for a few seconds (1, 2, 3, or 5 seconds) up to about 1, about 2, about 5, about 10, about 15, about 20, about 25, or about 30 minutes.

The cosmetic treatment composition may be applied to the hair immediately after or immediately before the hair it treated with another composition (e.g., a shampoo and/or a conditioner). For example, the hair-treatment compositions may be applied to the hair within about 1, 2, 5, 10, or 20 minutes before or after a shampoo and/or a conditioner is applied to the hair.

The cosmetic composition is unique in its ability to provide hair with improved manageability, long-lasting style and frizz control, and smoothness. Accordingly, the instant disclosure relates to methods for improving the manageability of hair, for imparting lasting style and frizz control, and for imparting smoothness. More specifically, the compositions may be used in methods for conditioning the hair, providing curl definition to the hair, providing frizz control to the hair, improving ease of combability and detangling of the hair, and providing smoothness to the hair.

### PREFERRED EMBODIMENTS

In various embodiments, the cosmetic composition comprises, consists essentially of, or consists of:
(a) about 5 to about 99 wt. %, preferably about 10 to about 60 wt. %, more preferably about 10 to about 40 wt. %, and even more preferably about 15 to about 25 wt. % of one or more alpha hydroxy acid esters;
(b) about 0.1 to about 10 wt. %, preferably about 0.5 to about 8 wt. %, more preferably about 1 to about 8 wt. %, and even more preferably, about 2 to about 6 wt. % of one or more fatty alcohols;
(c) about 0.1 to about 10 wt. %, preferably about 0.5 to about 8 wt. %, more preferably about 1 to about 6 wt. %, and even more preferably about 2 to about 5 wt. % of one or more cationic surfactants;
   provided that (b) and (c) are in a weight ratio of about 8:1 to about 1:5, preferably about 4:1 to about 1:2, more preferably about 3:1 to about 1:1;
(d) optionally, one or more fatty compounds, wherein the one or more fatty compounds is preferably in an amount of about 1 to about 90 wt. %, more preferably about 10 to about 85 wt. %, and more preferably about 25 to about 80 wt. %, and even more preferably about 50 to about 75 wt. %;
(e) optionally, about 0.01 to about 10 wt. %, preferably about 0.1 to about 8 wt. %, and more preferably about 1 to about 5 wt. % of one or more miscellaneous ingredients;
provided the composition is translucent or transparent;
the composition is essentially free from water, wherein water is preferably in an amount of less than 5 wt. %, more preferably less than 3 wt. %, and even more preferably less than 1 wt. %, and most preferably less than 0.1 wt. %;
the composition is essentially free from mono-alcohols having from 2 to 6 carbon atoms, wherein the mono-alcohols having from 2 to 6 carbon atoms are preferably in an amount of less than 5 wt. %, more preferably less than 3 wt. %, and even more preferably less than 1 wt. %, and most preferably less than 0.1 wt. %;
the composition is essentially free from polyols having from 2 to 8 carbon atoms, wherein the polyols having from 2 to 8 carbon atoms are preferably in an amount of less than 5 wt. %, more preferably less than 3 wt. %, and even more preferably less than 1 wt. %, and most preferably less than 0.1 wt. %; and
all percentages by weight are based on a total weight of the composition.

In a preferred embodiment, the cosmetic composition comprises, consists essentially of, or consists of:
(a) about 5 to about 99 wt. %, preferably about 10 to about 60 wt. %, more preferably about 10 to about 40 wt. %, and even more preferably about 15 to about 25 wt. % of one or more alpha hydroxy acid esters, wherein the alpha hydroxy acid of the alpha hydroxy acid esters are selected from glycolic acid, lactic acid, malic acid, tartaric acid, citric acid, or combinations thereof, preferably wherein:
   the glycolic acid ester has a formula of CH₂OH(COOR₅), and R₅ is C₂ to C₆ alkyl group;
   the lactic acid ester has a formula of C₂H₄OH(COOR₆), and Re is C₂ to C₆ alkyl group;
   the malic acid ester has a formula of C₂H₃OH(COOR₇)(COOR₈), R₇ and R₈ are independently H atom or C₂ to C₆ alkyl group, and one of R₇ and R₈ is an alkyl group;
   the tartaric acid ester has a formula of C₂H₂(OH)₂(COOR₉)(COOR₁₀), R₉ and R₁₀ are independently H atom or C₂ to C₆ alkyl group, and one of R₉ and R₁₀ is an alkyl group; and
   the citric acid ester has a formula of C₃H₄OH(COOR₁₁)(COOR₁₂)(COOR₁₃), R₁₁, R₁₂ and R₁₃ are independently H atom or C₂ to C₆ alkyl group, and one of R₁₁, R₁₂ and R₁₃ is an alkyl group;
(b) about 0.1 to about 10 wt. %, preferably about 0.5 to about 8 wt. %, more preferably about 1 to about 8 wt. %, and even more preferably, about 2 to about 6 wt. % of one or more fatty alcohols, wherein the one or more fatty alcohols are selected from fatty alcohol represented by R—OH, wherein R is a C₁₂-C₂₄ branched or straight alkyl or an alkenyl group, optionally substituted by one or more hydroxyl groups; wherein preferably, the one or more fatty alcohols are selected from 2-octyldodecanol, isostearyl alcohol, 2-hexyldecanol, 2-butyloctanol, 2-undecylpentadecanol, oleyl alcohol, linoleyl alcohol, linolenyl alcohol, undecylenyl alcohol, cetearyl alcohol, erucyl alcohol, stearyl alcohol, behenyl alcohol, arachidyl alcohols, or combinations thereof;
(c) about 0.1 to about 10 wt. %, preferably about 0.5 to about 8 wt. %, more preferably about 1 to about 6 wt. %, and even more preferably about 2 to about 5 wt. % of one or more cationic surfactants selected from quaternary ammonium surfactants and amidoamine surfactants, wherein:
   the one or more quaternary ammonium surfactants are selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, and tricetylmonium chloride, and
   the one or more amidoamine surfactants are selected from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamidopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, and stearamidoethyldiethylamine;
   provided that (b) and (c) are in a weight ratio of about 5:1 to about 1:5, preferably about 4:1 to about 1:2, more preferably about 3:1 to about 1:1;
(d) optionally, one or more fatty compounds, wherein the one or more fatty compounds are preferably in an amount of about 1 to about 90 wt. %, more preferably about 10 to about 85 wt. %, and more preferably about 25 to about 80 wt. %, and even more preferably about 50 to about 75 wt. %; wherein the one or more fatty compounds are preferably selected from propylene glycol dicaprylate/dicaprate, propylene glycol dicaprylate, dicaprylyl carbonate, dicaprylyl ether, dibutyl adipate, C12-15 alkyl benzoate, propylheptyl caprylate, ethylhexyl palmitate, coco-caprylate/caprate, butylene glycol dicaprylate/dicaprate, diisopropyl adipate, ethyl oleate, ethylhexyl hydroxystearate, glycol distearate, glycol stearate, ppg-20 methyl glucose ether distearate, triacetin, sucrose distearate, or combinations thereof; and/or the one or more fatty compounds are selected from fatty compounds having one ester group, fatty compounds having two ester groups, and fatty compounds having three ester groups, wherein:
   the fatty compounds having one ester group are selected from ethyl oleate, isopropyl myristate, isopropyl palmitate, isopropyl stearate, methyl laurate, methyl stearate, butyl oleate, cetyl stearate, stearyl alcohol acetate, butyl stearate, octyl stearate, isopropyl caprylate, isopropyl laurate, isopropyl linoleate, propylene glycol monolaurate, propylene glycol monopalmitate, glycerol monooleate, glycerol monostearate, methyl palmitate, butyl palmitate, cetyl palmitate, decyl oleate, octyl oleate, ethyl stearate, ethyl laurate, ethyl palmitate, ethyl caprylate, ethyl decanoate, butyl caprate, octyl decanoate, methyl decanoate, butyl myristate, hexyl myristate, isocetyl stearate, isostearyl alcohol acetate, isopropyl caprate, neopentyl glycol monolaurate, neopentyl glycol monopalmitate, diethylhexyl laurate, propylene glycol monostearate, butyl laurate, stearyl alcohol caprate, glyceryl monostearate, glyceryl monopalmitate, ethylcaprylate, butyl stearate, diisopropyl oleate, diethyl hexanoate, methyl hexanoate, ethyl hexanoate, diisopropyl linoleate, isopropyl stearoyl stearate, butylene glycol monolaurate, butylene glycol monostearate, isopropyl myristate, isopropyl arachidonate, decyl palmitate, and cetyl caprate,
   the fatty compounds having two ester groups are selected from diethyl succinate, dibutyl succinate, diethylhexyl succinate, diisopropyl sebacate, diethyl sebacate, dibutyl sebacate, diethylhexyl malate, diethylhexyl maleate, dicapryl adipate, dicaprylyl maleate, neopentyl glycol dicaprate, propylene glycol dicaprylate/dicaprate, neopentyl glycol diisostearate, neopentyl glycol diethylhexanoate, neopentyl glycol diheptanoate, diisopropyl adipate, diisopropyl dilaurate, dipropylene glycol dicaprylate, diethylhexyl adipate, diethylhexyl caprate, diisobutyl caprate, neopentyl glycol dipalmitate, ethylhexyl caprate, ethylhexyl caprylate, isopropyl caprate, butylene glycol dicaprylate, butylene glycol dicaprate, dipropylene glycol dicaprate, methyl diethylsuccinate, propylene glycol dodecylbenzoate, methyl ethylcaprylate, propylene glycol laurate, octyl dodecylbenzoate, diethoxyethyl hexanoate, ethylhexyl hexanoate, isononyl isononanoate, diethylene glycol dipropionate, propylene glycol dicaprylate, dimethyl adipate, diethyl isononanoate, diisopropyl dilinoleate, dimethyl sebacate, ethyl hexanoate, diisopropyl octanoate, isopropyl dicaprylate, propyl palmitate, ethyl stearate, diethyl decanoate, decyl adipate, diisopropyl stearate, methyl palmitate, methyl laurate, butyl laurate, diethyl myristate, hexyl laurate, ethyl esters of fatty acids, isopropyl myristate, diacetylated propylene glycol, butyl oleate, isopropyl stearate, butyl myristate, diisobutyl adipate, ethyl oleate, cetyl stearate, polyol esters of fatty acids, ethyl decanoate, ethyl hexyl stearate, diethyl stearate, butyl caprylate, methyl ester of lauric acid, hexyl caprate, ethylcaprylate, and ethyl caprylate, and
   the fatty compounds having three ester groups are selected from glycerol trioleate, glycerol tricaprylate, glycerol tricaprate, glycerol tristearate, glycerol trilaurate, glycerol triacetate, triethyl citrate, triisostearyl citrate, trimethylolpropane trioleate, trimethylolpropane tricaprylate, trimethylolpropane tricitrate, trimethylolpropane trioctanoate, trimethylolpropane tripalmitate, triacetin, triglyceride of caprylic acid, triglyceride of capric acid, triglyceride of stearic acid, triglyceride of lauric acid, triglyceride of oleic acid, tri-n-butyl citrate, tri-n-octyl citrate, trimethylolpropane triisostearate, triacetylglycerol, glyceryl trioleate, glyceryl trilinoleate, trimethylolpropane trisunflower oil, glyceryl tricaprylate, glyceryl tricaprate, glyceryl tristearate, glyceryl triarachidonate, glyceryl trilaurate, triglyceride of linoleic acid, triethylhexanoin, and triheptanoin;
(e) optionally, about 0.01 to about 10 wt. %, preferably about 0.1 to about 8 wt. %, and more preferably about 1 to about 5 wt. % of one or more miscellaneous ingredients, wherein the one or more miscellaneous ingredients are preferably selected from preservatives, fragrances, pH adjusters, salts, chelating agents, buffers, composition colorants, fillers, proteins, peptides, protein hydrolysates and lysates, vitamins, botanical extracts, and a combination thereof;
   provided the composition is translucent or transparent;
   the composition is essentially free from water, wherein water is preferably in an amount of less than 5 wt. %, more preferably less than 3 wt. %, and even more preferably less than 1 wt. %, and most preferably less than 0.1 wt. %;
   the composition is essentially free from mono-alcohols having from 2 to 6 carbon atoms, wherein the mono-alcohols having from 2 to 6 carbon atoms are preferably in an amount of less than 5 wt. %, more preferably less than 3 wt. %, and even more preferably less than 1 wt. %, and most preferably less than 0.1 wt. %;
   the composition is essentially free from polyols having from 2 to 8 carbon atoms, wherein the polyols having from 2 to 8 carbon atoms are preferably in an amount of less than 5 wt. %, more preferably less than 3 wt. %, and even more preferably less than 1 wt. %, and most preferably less than 0.1 wt. %; and
   all percentages by weight are based on a total weight of the composition.

In a preferred embodiment, the cosmetic composition comprises, consists essentially of, or consists of:
(a) about 5 to about 99 wt. %, preferably about 10 to about 60 wt. %, more preferably about 10 to about 40 wt. %, and even more preferably about 15 to about 25 wt. % of one or more alpha hydroxy acid esters selected from esters of glycolic acid, lactic acid, malic acid, tartaric acid, citric acid, or combinations thereof, wherein:
   the glycolic acid ester has a formula of CH₂OH(COOR₅), and R₅ is C₂ to C₆ alkyl group, and is preferably selected from methyl glycolate, ethyl glycolate, n-propyl glycolate, isopropyl glycolate, butyl glycolate, or combinations thereof;
   the lactic acid ester has a formula of C₂H₄OH(COOR₆), and R₆ is C₂ to C₆ alkyl group, and is preferably selected from methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, or combinations thereof;
   the malic acid ester has a formula of C₂H₃OH(COOR₇)(COOR₈), R₇ and R₈ are independently H atom or C₂ to C₆ alkyl group, and one of R₇ and R₈ is an alkyl group, and is preferably selected from dimethyl malate, diethyl malate, diisopropyl malate, dipropyl malate, dibutyl malate, diisobutyl malate, ditertbutyl malate, dimethyl acetyl malate, diethyl acetyl malate, dipropyl acetyl malate, diisopropyl acetyl malate, dibutyl acetyl malate, diisobutyl acetyl malate, ditertbutyl acetyl malate, or combinations thereof;
   the tartaric acid ester has a formula of C₂H₂(OH)₂(COOR₉)(COOR₁₀), R₉ and R₁₀ are independently H atom or C₂ to C₆ alkyl group, and one of R₉ and R₁₀ is an alkyl group, and is preferably selected from dimethyl tartrate, diethyl tartrate, dibutyl tartrate, diisopropyl tartrate, or combinations thereof; and
   the citric acid ester has a formula of C₃H₄OH(COOR₁₁)(COOR₁₂)(COOR₁₃), R₁₁, R₁₂ and R₁₃ are independently H atom or C₂ to C₆ alkyl group, and one of R₁₁, R₁₂ and R₁₃ is an alkyl group, and is preferably selected from trimethyl citrate, triethyl citrate, tri-n-butyl citrate, tributyl citrate, acetyl triethyl citrate, acetyl tributyl citrate, trihexyl citrate, butyl trihexyl citrate, stearyl citrate, ethyl mandelate, ethyl salicylate, methyl salicylate, ethyl glycolate, or combinations thereof;
(b) about 0.1 to about 10 wt. %, preferably about 0.5 to about 8 wt. %, more preferably about 1 to about 8 wt. %, and even more preferably, about 2 to about 6 wt. % of one or more fatty alcohols, wherein the one or more fatty alcohols are selected from fatty alcohol represented by R—OH, wherein R is a C₁₂-C₂₄ branched or straight alkyl or an alkenyl group, optionally substituted by one or more hydroxyl groups; wherein preferably, the one or more fatty alcohols are selected from 2-octyldodecanol, isostearyl alcohol, 2-hexyldecanol, 2-butyloctanol, 2-undecylpentadecanol, oleyl alcohol, linoleyl alcohol, linolenyl alcohol, undecylenyl alcohol, cetearyl alcohol, erucyl alcohol, stearyl alcohol, behenyl alcohol, arachidyl alcohols, or combinations thereof;
(c) about 0.1 to about 10 wt. %, preferably about 0.5 to about 8 wt. %, more preferably about 1 to about 6 wt. %, and even more preferably about 2 to about 5 wt. % of one or more cationic surfactants selected from quaternary ammonium surfactants and amidoamine surfactants, wherein:
   the one or more quaternary ammonium surfactants are selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, and tricetylmonium chloride, and
   the one or more amidoamine surfactants are selected from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamidopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, and stearamidoethyldiethylamine;
   wherein preferably, at least one or all of the one or more cationic surfactants are selected from the amidoamine surfactants;
   provided that (b) and (c) are in a weight ratio of about 8:1 to about 1:5, preferably about 6:1 to about 1:2, more preferably about 3:1 to about 1:1;
(d) about 10 to about 85 wt. %, preferably about 50 to about 80 wt. %, and more preferably about 60 to about 75 wt. % of one or more fatty compounds, wherein the one or more fatty compounds are preferably selected from propylene glycol dicaprylate/dicaprate, propylene glycol dicaprylate, dicaprylyl carbonate, dicaprylyl ether, dibutyl adipate, C12-15 alkyl benzoate, propylheptyl caprylate, ethylhexyl palmitate, coco-caprylate/caprate, butylene glycol dicaprylate/dicaprate, diisopropyl adipate, ethyl oleate, ethylhexyl hydroxystearate, glycol distearate, glycol stearate, ppg-20 methyl glucose ether distearate, triacetin, sucrose distearate, or combinations thereof; and/or the one or more fatty compounds are selected from fatty compounds having one ester group and fatty compounds having two ester groups, wherein:
   the fatty compounds having one ester group are selected from ethyl oleate, isopropyl myristate, isopropyl palmitate, isopropyl stearate, methyl laurate, methyl stearate, butyl oleate, cetyl stearate, stearyl alcohol acetate, butyl stearate, octyl stearate, isopropyl caprylate, isopropyl laurate, isopropyl linoleate, propylene glycol monolaurate, propylene glycol monopalmitate, glycerol monooleate, glycerol monostearate, methyl palmitate, butyl palmitate, cetyl palmitate, decyl oleate, octyl oleate, ethyl stearate, ethyl laurate, ethyl palmitate, ethyl caprylate, ethyl decanoate, butyl caprate, octyl decanoate, methyl decanoate, butyl myristate, hexyl myristate, isocetyl stearate, isostearyl alcohol acetate, isopropyl caprate, neopentyl glycol monolaurate, neopentyl glycol monopalmitate, diethylhexyl laurate, propylene glycol monostearate, butyl laurate, stearyl alcohol caprate, glyceryl monostearate, glyceryl monopalmitate, ethylcaprylate, butyl stearate, diisopropyl oleate, diethyl hexanoate, methyl hexanoate, ethyl hexanoate, diisopropyl linoleate, isopropyl stearoyl stearate, butylene glycol monolaurate, butylene glycol monostearate, isopropyl myristate, isopropyl arachidonate, decyl palmitate, and cetyl caprate, and
   the fatty compounds having two ester groups are selected from diethyl succinate, dibutyl succinate, diethylhexyl succinate, diisopropyl sebacate, diethyl sebacate, dibutyl sebacate, diethylhexyl malate, diethylhexyl maleate, dicapryl adipate, dicaprylyl maleate, neopentyl glycol dicaprate, propylene glycol dicaprylate/dicaprate, neopentyl glycol diisostearate, neopentyl glycol diethylhexanoate, neopentyl glycol diheptanoate, diisopropyl adipate, diisopropyl dilaurate, dipropylene glycol dicaprylate, diethylhexyl adipate, diethylhexyl caprate, diisobutyl caprate, neopentyl glycol dipalmitate, ethylhexyl caprate, ethylhexyl caprylate, isopropyl caprate, butylene glycol dicaprylate, butylene glycol dicaprate, dipropylene glycol dicaprate, methyl diethylsuccinate, propylene glycol dodecylbenzoate, methyl ethylcaprylate, propylene glycol laurate, octyl dodecylbenzoate, diethoxyethyl hexanoate, ethylhexyl hexanoate, isononyl isononanoate, diethylene glycol dipropionate, propylene glycol dicaprylate, dimethyl adipate, diethyl isononanoate, diisopropyl dilinoleate, dimethyl sebacate, ethyl hexanoate, diisopropyl octanoate, isopropyl dicaprylate, propyl palmitate, ethyl stearate, diethyl decanoate, decyl adipate, diisopropyl stearate, methyl palmitate, methyl laurate, butyl laurate, diethyl myristate, hexyl laurate, ethyl esters of fatty acids, isopropyl myristate, diacetylated propylene glycol, butyl oleate, isopropyl stearate, butyl myristate, diisobutyl adipate, ethyl oleate, cetyl stearate, polyol esters of fatty acids, ethyl decanoate, ethyl hexyl stearate, diethyl stearate, butyl caprylate, methyl ester of lauric acid, hexyl caprate, ethylcaprylate, and ethyl caprylate;
   wherein the one or more fatty compounds are more preferably selected from propylene glycol dicaprylate/dicaprate, propylene glycol dicaprylate, dicaprylyl carbonate, dicaprylyl ether, or combinations thereof; and wherein the one or more fatty compounds is most preferably propylene glycol dicaprylate/dicaprate; and
(e) optionally, about 0.01 to about 10 wt. %, preferably about 0.1 to about 8 wt. %, and more preferably about 1 to about 5 wt. % of one or more miscellaneous ingredients, wherein the one or more miscellaneous ingredients are preferably selected from preservatives, fragrances, pH adjusters, salts, chelating agents, buffers, composition colorants, fillers, proteins, peptides, protein hydrolysates and lysates, vitamins, botanical extracts, and a combination thereof;
provided the composition is translucent or transparent;
the composition is preferably free or essentially free from dicaprylyl ether, dicaprylyl carbonate, isopropylidene glycerol, or combinations thereof;
the composition is essentially free from water, wherein water is preferably in an amount of less than 2 wt. %, more preferably less than 1 wt. %, and even more preferably less than 0.5 wt. %, and most preferably less than 0.1 wt. %;
the composition is essentially free from mono-alcohols having from 2 to 6 carbon atoms, wherein the mono-alcohols having from 2 to 6 carbon atoms are preferably in an amount of less than 2 wt. %, more preferably less than 1 wt. %, and even more preferably less than 0.5 wt. %, and most preferably less than 0.1 wt. %;
the composition is essentially free from polyols having from 2 to 8 carbon atoms, wherein the polyols having from 2 to 8 carbon atoms are preferably in an amount less than 2 wt. %, more preferably less than 1 wt. %, and even more preferably less than 0.5 wt. %, and most preferably less than 0.1 wt. %; and
all percentages by weight are based on a total weight of the composition.

In a preferred embodiment, the cosmetic composition comprises, consists essentially of, or consists of:
(a) about 5 to about 40 wt. %, preferably about 10 to about 30 wt. %, more preferably about 15 to about 25 wt. % of one or more alpha hydroxy acid esters selected from diethyl tartrate, ethyl lactate, and triethyl citrate;
(b) about 0.1 to about 10 wt. %, preferably about 0.5 to about 8 wt. %, more preferably about 1 to about 8 wt. %, and even more preferably, about 2 to about 6 wt. % of one or more fatty alcohols, wherein the one or more fatty alcohols are selected from fatty alcohol represented by R—OH, wherein R is a C₁₂-C₂₄ branched or straight alkyl or an alkenyl group, optionally substituted by one or more hydroxyl groups; wherein preferably, the one or more fatty alcohols are selected from 2-octyldodecanol, isostearyl alcohol, 2-hexyldecanol, 2-butyloctanol, 2-undecylpentadecanol, oleyl alcohol, linoleyl alcohol, linolenyl alcohol, undecylenyl alcohol, cetearyl alcohol, erucyl alcohol, stearyl alcohol, behenyl alcohol, arachidyl alcohols, or combinations thereof;
(c) about 0.1 to about 10 wt. %, preferably about 0.5 to about 8 wt. %, more preferably about 1 to about 6 wt. %, and even more preferably about 2 to about 5 wt. % of one or more cationic surfactants selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, and tricetylmonium chloride, oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamidopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, and stearamidoethyldiethylamine, wherein the one or more cationic surfactants are preferably selected from cetrimonium chloride, stearimonium chloride, behentrimonium chloride, behentrimonium methosulfate, distearyldimonium chloride, oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, and behenamidopropyl dimethylamine;
   provided that (b) and (c) are in a weight ratio of about 8:1 to about 1:4, preferably about 4:1 to about 1:2, more preferably about 2:1 to about 1:1;
(d) about 50 to about 85 wt. %, preferably about 60 to about 80 wt. %, and more preferably about 65 to about 75 wt. % of one or more fatty compounds, wherein the one or more fatty compounds are preferably selected from propylene glycol dicaprylate/dicaprate, propylene glycol dicaprylate/dicaprate, dicaprylyl carbonate, dicaprylyl ether, dibutyl adipate, C12-15 alkyl benzoate, propylheptyl caprylate, ethylhexyl palmitate, coco-caprylate/caprate, butylene glycol dicaprylate/dicaprate, diisopropyl adipate, ethyl oleate, ethylhexyl hydroxystearate, glycol distearate, glycol stearate, PPG-20 methyl glucose ether distearate, triacetin, sucrose distearate, or combinations thereof; wherein more preferably, the one or more fatty compounds are selected from propylene glycol dicaprylate/dicaprate, dicaprylyl carbonate, dicaprylyl ether, or combinations thereof;
   wherein the one or more fatty compounds are more preferably selected from propylene glycol dicaprylate/dicaprate, propylene glycol dicaprylate, dicaprylyl carbonate, dicaprylyl ether, or combinations thereof; and wherein the one or more fatty compounds is most preferably propylene glycol dicaprylate/dicaprate;
(e) optionally, about 0.01 to about 10 wt. %, preferably about 0.1 to about 8 wt. %, and more preferably about 1 to about 5 wt. % of one or more miscellaneous ingredients, wherein the one or more miscellaneous ingredients are preferably selected from preservatives, fragrances, pH adjusters, salts, chelating agents, buffers, composition colorants, fillers, proteins, peptides, protein hydrolysates and lysates, vitamins, botanical extracts, and a combination thereof;
provided the composition is translucent or transparent;
the composition is essentially free from water, wherein water is preferably in an amount of less than 2 wt. %, more preferably less than 1 wt. %, and even more preferably less than 0.5 wt. %, and most preferably less than 0.1 wt. %;
the composition is essentially free from mono-alcohols having from 2 to 6 carbon atoms, wherein the mono-alcohols having from 2 to 6 carbon atoms are preferably in an amount of less than 2 wt. %, more preferably less than 1 wt. %, and even more preferably less than 0.5 wt. %, and most preferably less than 0.1 wt. %;
the composition is essentially free from polyols having from 2 to 8 carbon atoms, wherein the polyols having from 2 to 8 carbon atoms are preferably in an amount less than 2 wt. %, more preferably less than 1 wt. %, and even more preferably less than 0.5 wt. %, and most preferably less than 0.1 wt. %; and
all percentages by weight are based on a total weight of the composition.

In a preferred embodiment, the cosmetic composition comprises, consists essentially of, or consists of:
(a) about 5 to about 40 wt. %, preferably about 10 to about 30 wt. %, more preferably about 15 to about 25 wt. % of one or more alpha hydroxy acid esters selected from diethyl tartrate, ethyl lactate, and triethyl citrate;
(b) about 0.1 to about 10 wt. %, preferably about 0.5 to about 8 wt. %, more preferably about 1 to about 8 wt. %, and even more preferably, about 2 to about 6 wt. % of one or more fatty alcohols, wherein the one or more fatty alcohols are selected from fatty alcohol represented by R—OH, wherein R is a C₁₂-C₂₄ branched or straight alkyl or an alkenyl group, optionally substituted by one or more hydroxyl groups; wherein preferably, the one or more fatty alcohols are selected from 2-octyldodecanol, isostearyl alcohol, 2-hexyldecanol, 2-butyloctanol, 2-undecylpentadecanol, oleyl alcohol, linoleyl alcohol, linolenyl alcohol, undecylenyl alcohol, cetearyl alcohol, erucyl alcohol, stearyl alcohol, behenyl alcohol, arachidyl alcohols, or combinations thereof;
(c) about 0.1 to about 10 wt. %, preferably about 0.5 to about 8 wt. %, more preferably about 1 to about 6 wt. %, and even more preferably about 2 to about 5 wt. % of one or more cationic surfactants selected from cetrimonium chloride, stearimonium chloride, behentrimonium chloride, behentrimonium methosulfate, behenamidopropyltrimonium methosulfate, stearamidopropyltrimonium chloride, stearamidopropyl dimethylamine, isostearamidopropyl dimethylamine;
   provided that (b) and (c) are in a weight ratio of about 8:1 to about 1:4, preferably about 4:1 to about 1:2, more preferably about 2:1 to about 1:1;
(d) about 50 to about 85 wt. %, preferably about 60 to about 80 wt. %, and more preferably about 65 to about 75 wt. % of propylene glycol dicaprylate/dicaprate;
(e) optionally, about 0.01 to about 10 wt. %, preferably about 0.1 to about 8 wt. %, and more preferably about 1 to about 5 wt. % of one or more miscellaneous ingredients, wherein the one or more miscellaneous ingredients are preferably selected from preservatives, fragrances, pH adjusters, salts, chelating agents, buffers, composition colorants, fillers, proteins, peptides, protein hydrolysates and lysates, vitamins, botanical extracts, and a combination thereof;
provided the composition is translucent or transparent;
the composition is essentially free from water, wherein water is preferably in an amount of less than 2 wt. %, more preferably less than 1 wt. %, and even more preferably less than 0.5 wt. %, and most preferably less than 0.1 wt. %;
the composition is essentially free from mono-alcohols having from 2 to 6 carbon atoms, wherein the mono-alcohols having from 2 to 6 carbon atoms are preferably in an amount of less than 2 wt. %, more preferably less than 1 wt. %, and even more preferably less than 0.5 wt. %, and most preferably less than 0.1 wt. %;
the composition is essentially free from polyols having from 2 to 8 carbon atoms, wherein the polyols having from 2 to 8 carbon atoms are preferably in an amount less than 2 wt. %, more preferably less than 1 wt. %, and even more preferably less than 0.5 wt. %, and most preferably less than 0.1 wt. %; and
all percentages by weight are based on a total weight of the composition.

### EXAMPLES

Various changes can be made in the above-described compositions and methods without departing from the scope of the invention. Accordingly, it is intended that all disclosure contained in the above description and in the examples given below, shall be interpreted as illustrative and not in a limiting sense.

### Example 1

### Inventive Compositions

| | | | A | B | C |
|---|---|---|---|---|---|
| (a) | Alpha | DIETHYL TARTRATE | | | 20 |
| | Hydroxy Acid | ETHYL LACTATE | | 20 | |
| | Ester | TRIETHYL CITRATE | 20 | | |
| (b) | Fatty Alcohol | OLEYL ALCOHOL | 5 | 5 | 5 |
| (c) | Cationic | STEARAMIDOPROPYL | 2.8 | 2.8 | 2.8 |
| | Surfactant | DIMETHYLAMINE | | | |
| Weight Ratio of (b):(c) | | | 1.8:1 | 1.8:1 | 1.8:1 |
| (d) | Fatty | PROPYLENE GLYCOL | 72.2 | 72.2 | 72.2 |
| | Compound | DICAPRYLATE/ | | | |
| | | DICAPRATE | | | |

### Example 2

### Comparative Testing

| | | Inventive | | | Comparative | | |
|---|---|---|---|---|---|---|---|
| | | A | B | C | C-1 | C-2 | C-3 |
| Alpha | DIETHYL TARTRATE | | | 20 | | 20 | 20 |
| Hydroxy | ETHYL LACTATE | | 20 | | | | |
| Acid Ester | TRIETHYL CITRATE | 20 | | | | | |
| | DIETHYLHEXYL | | | | | | |
| | MALEATE | | | | | | |
| Fatty | OLEYL ALCOHOL | 5 | 5 | 5 | 5 | | 5 |
| Alcohol | | | | | | | |
| Cationic | STEARAMIDOPROPYL | 2.8 | 2.8 | 2.8 | 2.8 | 2.8 | |
| Surfactant | DIMETHYLAMINE | | | | | | |
| Fatty | PROPYLENE GLYCOL | 72.2 | 72.2 | 72.2 | 92.2 | 97.2 | 95 |
| Compound | DICAPRYLATE/ | | | | | | |
| | DICAPRATE | | | | | | |

| | | | | | | |
|---|---|---|---|---|---|---|
| Solubilized | Y | Y | Y | N | N | Y |
| Lamellar Phase | Y | Y | Y | N | N | N |

Y = Yes
N = No

Comparative compositions C-1 through C-3 are like inventive composition C except that the alpha hydroxy acid ester (diethyl tartrate) is absent from comparative composition C-1, the fatty alcohol (oleyl alcohol) is absent from composition C-2, and the cationic surfactant (stearamidopropyl dimethylamine) is absent from composition C-3. Without the alpha hydroxy acid ester, composition C-1 was not fully solubilized. Without the fatty alcohol, composition C-2 also was not fully solubilized. When the alpha hydroxy acid ester, the fatty alcohol, or the cationic surfactant is omitted, the compositions fail to form a lamellar structure, as shown by compositions C-1, C-2, and C-3.

Testing was carried out to determine how inventive compositions A-C and comparative compositions C-3 perform. SA 20 regular bleached Caucasian hair swatches, curl type 3A-3B (Andre Walker curl system) were obtained and initially washed with a standard shampoo. After rinsing, one swatch was set aside for use as a control. The control swatch was simply hung to dry after cleansing with the standard shampoo. After rinsing the shampoo from the remaining hair swatches, the inventive and comparative compositions were applied, and massaged into the hair swatches (0.4 grams/gram of swatch), and allowed to remain on the hair for 10 minutes. After 10 minutes, the swatches were rinsed with water, and hung to dry. After all hair swatches were dry, they were visually examined to determined softness, smoothness, alignment, and frizz control (lack of frizz). The hair swatch treated with inventive compositions A-C were soft and smooth and exhibited the better fiber alignment and frizz control (lack of frizz) than comparative composition C-3. All compositions performed better than the control. Images of the hair swatches are provided in the FIGURE.

The terms “comprising,” “having,” and “including” are used in their open, non-limiting sense.

The terms “a” and “the” are understood to encompass the plural as well as the singular.

All percentages, parts and ratios herein are based upon the total weight of the compositions of the present disclosure, unless otherwise indicated.

The compositions and methods of the present disclosure can comprise, consist of, or consist essentially of the essential elements and limitations of the disclosure described herein, as well as any additional or optional ingredients, components, or limitations described herein or otherwise useful.

The term “rinse-off” as used herein indicates that the composition is used in a context whereby the composition is ultimately rinsed or washed from the treated surface, (e.g., skin, hair, or hard surfaces) either after or during the application of the product. These rinse-off compositions are to be distinguished from compositions referred to a “leave-on” compositions. For example, a rinse-off composition is applied to the hair, optionally allowed to remain on the hair for a short time (e.g., a few second up to about 5, 10, or 15 minutes) and subsequently rinsed from the hair before the hair styled.

The compositions described throughout this disclosure may be a “leave-on” composition. A “leave-on” (also called leave-in) composition refers to a composition that is applied to skin or hair and is not subjected to immediate rinsing and/or washing, for example for at least 4 hours or for a period of time ranging from 4 hours up to 72 hours, from 4 hours up to 48 hours, or from 8 hours up to 36 hours, or from 8 hours up to 24 hours. In other words, the product is applied to the hair and remains on the hair, as styled, i.e., it is not removed from the hair prior to styling the hair.

A “lamellar phase” refers generally to packing of polar-headed long chain nonpolar-tail molecules in an environment of bulk polar liquid (i.e., water from the hair), as sheets of bilayers separated by bulk liquid.

The term “non-linear fatty acids” as used in the instant disclosure refers to unsaturated fatty acid and/or branched fatty acids.

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition.

The term “translucent” with respect to a translucent composition indicates that the composition has a transmittance of at least 50% at a wavelength of 600 nm, for example measured using a Lambda 40 UV-visible spectrometer. A human cannot likely see through a translucent composition, for example, and read the text on the other side of a clear glass or clear plastic bottle containing the composition. Rather, the text is usually blurred and difficult or not possible to read, yet movement and structure can normally be identified.

The term “opaque” with respect to an opaque composition indicates that the composition is not transparent or translucent, i.e., has a transmittance of less than 50% at a wavelength of 600 nm, for example measured using a Lambda 40 UV-visible spectrometer.

A “composition colorant” is a compound or ingredient that colors the composition but does not have an appreciable coloring effect on hair. In other words, the composition colorant is included to provide a color to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair. Thus, a composition colorant is different from a hair coloring agent. A composition colorant colors the compositions; a “hair coloring agent,” on the other hand, may change the color of the compositions (and often does) but also changes the color of hair

All ranges and values disclosed herein are inclusive and combinable. For examples, any value or point described herein that falls within a range described herein can serve as a minimum or maximum value to derive a sub-range. Furthermore, all ranges provided are meant to include every specific range within, and combination of sub-ranges between, the given ranges. Thus, a range from 1-5, includes specifically 1, 2, 3, 4 and 5, as well as sub-ranges such as 2-5, 3-5, 2-3, 2-4, 1-4, etc.

The expression “inclusive” for a range means that the limits of the range are included in the defined interval.

Other than in the operating examples, or where otherwise indicated, all numbers expressing quantities of ingredients and/or reaction conditions are to be understood as being modified in all instances by the term “about,” meaning within +/−10% of the indicated number. Thus, and amount of “about 5 wt. %” includes amounts from 4.5 to 5.5 wt. %. An amount of “about 65 wt. %” includes amounts from 58.5 to 71.5 wt. %.

As used herein, the expression “at least one” is interchangeable with the expression “one or more” and thus includes individual components as well as mixtures/combinations.

When referencing amounts “less than” a specified value, the amount includes zero (none). For example, an amount of “less than 5 wt. %” of a specific material means that the specific material may be present, provided it is present in an amount that is below 5 wt. %, or may be absent.

The term “substantially free” or “essentially free” as used herein means that there is less than about 5 wt. % of a specific material added to a composition, based on the total weight of the compositions. Nonetheless, the compositions may include less than about 2 wt. %, less than about 1 wt. %, less than about 0.5 wt. %, less than about 0.1 wt. %, less than 0.01 wt. %, or none of the specified material. For instance, the compositions of the instant disclosure are “essentially anhydrous.” Therefore, the composition includes no water or an amount of water less than about 5 wt. %. Nonetheless, the compositions may include less than about 2 wt. %, less than about 1 wt. %, less than about 0.5 wt. %, less than about 0.1 wt. %, less than 0.01 wt. %, or no water.

The term “active material” as used herein with respect to the percent amount of an ingredient or raw material, refers to 100% activity of the ingredient or raw material. All ingredients are listed as 100% active material unless otherwise indicated.

“Cosmetically acceptable” means that the item in question is compatible with a keratinous substrate such as skin and hair. For example, a “cosmetically acceptable carrier” means a carrier that is compatible with a keratinous substrate such as skin and hair.

Throughout the disclosure, the term “a combination thereof” (or a mixture thereof) may be used following a list of elements as shown in the following example where letters A-F represent the elements: “one or more elements selected from A, B, C, D, E, F, or a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is like the phrase “one or more elements selected from A, B, C, D, E, F, and a combination of any two or more of A, B, C, D, E, and F.”

The term “a salt thereof” also relates to “salts thereof.” Thus, where the disclosure refers to “an element selected from A, B, C, D, E, F, a salt thereof, or a combination thereof,” it indicates that that one or more of A, B, C, D, and F may be included, one or more of a salt of A, a salt of B, a salt of C, a salt of D, a salt of E, and a salt of F may be included, or a combination of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is nonlimiting.

Some of the compounds discussed throughout the disclosure may be in the form of a salt in the composition or added to the composition in the form of a salt (and may dissociate in the composition). Thus, all compounds and amounts of compounds set forth throughout this disclosure relate to both the salt form of the compound and to the disassociated form of the compound. In other words, even if the expression “a salt thereof” is not specifically or expressly stated with respect to ingredients that can form salts or are available as salts, it is understood that the salt form of the compound is encompassed.

“Volatile”, as used herein, means having a flash point of less than about 100° C.

“Non-volatile”, as used herein, means having a flash point of greater than about 100° C.

The term “polymers,” as defined herein, include homopolymers and copolymers formed from at least two different types of monomers.

The term “INCI” is an abbreviation of International Nomenclature of Cosmetic Ingredients, which is a system of names provided by the International Nomenclature Committee of the Personal Care Products Council to describe personal care ingredients.

All components and elements positively set forth in this disclosure can be negatively excluded from the claims. In other words, the compositions of the instant disclosure can be free or essentially free of any components and elements positively recited throughout the instant disclosure.

Some of the various categories of components identified may overlap. In such cases where overlap may exist and the composition includes both components (or the composition includes more than two components that overlap), an overlapping component or ingredient does not represent more than one element of the claims. For example, if a polyacrylate falls within the description of a thickening polymer and within the description of film forming polymer, a single polyacrylate is understood as being only the thickening polymer or only the film forming polymer. A single polyacrylate cannot simultaneously be construed as both a thickening polymer and a film forming polymer in the same composition, for example, when the claims set forth both a thickening polymer and a film forming polymer (even though the polyacrylate may function as both a thickening polymer and a film forming polymer). In other words, a single compound or ingredient cannot simultaneously serve as two different components or elements of a claim.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
(a) about 5 to about 99 wt. % of one or more alpha hydroxy acid esters;
(b) about 0.1 to about 10 wt. % of one or more fatty alcohols;
(c) about 0.1 to about 10 wt. % of one or more cationic surfactants; and
(d) optionally, one or more fatty compounds;
provided the composition is translucent or transparent;
the composition is essentially free from water, mono-alcohols having from 2 to 6 carbon atoms, and polyols having from 2 to 8 carbon atoms, and
all percentages by weight are based on a total weight of the composition.

2. The composition of claim 1, wherein the alpha hydroxy acid is selected from glycolic acid, lactic acid, malic acid, tartaric acid, citric acid, or combinations thereof.

3. The composition of claim 2, wherein
the glycolic acid ester has a formula of CH₂OH(COOR₅), and R₅ is C₂ to C₆ alkyl group;
the lactic acid ester has a formula of C₂H₄OH(COOR₆), and Re is C₂ to C₆ alkyl group;
the malic acid ester has a formula of C₂H₃OH(COOR₇)(COOR₈), R₇ and R₈ are independently H atom or C₂ to C₆ alkyl group, and one of R₇ and R₈ is an alkyl group;
the tartaric acid ester has a formula of C₂H₂(OH)₂(COOR₉)(COOR₁₀), R₉ and R₁₀ are independently H atom or C₂ to C₆ alkyl group, and one of R₉ and R₁₀ is an alkyl group; and
the citric acid ester has a formula of C₃H₄OH(COOR₁₁)(COOR₁₂)(COOR₁₃), R₁₁, R₁₂ and R₁₃ are independently H atom or C₂ to C₆ alkyl group, and one of R₁₁, R₁₂ and R₁₃ is an alkyl group.

4. The composition of claim 2, wherein
the glycolic acid ester is selected from methyl glycolate, ethyl glycolate, n-propyl glycolate, isopropyl glycolate, butyl glycolate, or combinations thereof;
the lactic acid ester is selected from methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, or combinations thereof;
the malic acid ester is selected from dimethyl malate, diethyl malate, diisopropyl malate, dipropyl malate, dibutyl malate, diisobutyl malate, ditertbutyl malate, dimethyl acetyl malate, diethyl acetyl malate, dipropyl acetyl malate, diisopropyl acetyl malate, dibutyl acetyl malate, diisobutyl acetyl malate, ditertbutyl acetyl malate, or combinations thereof;
the tartaric acid ester is selected from dimethyl tartrate, diethyl tartrate, dibutyl tartrate, diisopropyl tartrate, or combinations thereof; and
the citric acid ester is selected from trimethyl citrate, triethyl citrate, tri-n-butyl citrate, tributyl citrate, acetyl triethyl citrate, acetyl tributyl citrate, trihexyl citrate, butyl trihexyl citrate, stearyl citrate, ethyl mandelate, ethyl salicylate, methyl salicylate, ethyl glycolate, or combinations thereof.

5. The composition of claim 3, wherein the alpha hydroxy acid esters are selected from diethyl tartrate, ethyl lactate, triethyl citrate, or combinations thereof.

6. The composition of claim 1, wherein the one or more fatty alcohols are selected from fatty alcohol represented by R—OH, wherein R is a C₁₂-C₂₄ branched or straight alkyl or an alkenyl group, optionally substituted by one or more hydroxyl groups.

7. The composition of claim 6, wherein the one or more fatty alcohols are selected from lauryl alcohol, myristyl alcohol, cetyl alcohol, stearyl alcohol, oleyl alcohol, cetearyl alcohol, isostearyl 2-octyldodecanol, isostearyl alcohol, 2-hexyldecanol, 2-butyloctanol, 2-undecylpentadecanol, linoleyl alcohol, linolenyl alcohol, undecylenyl alcohol, erucyl alcohol, behenyl alcohol, arachidyl alcohols, or combinations thereof.

8. The composition of claim 1, wherein the one or more cationic surfactants are selected from quaternary ammonium surfactants, amidoamine surfactants, or combinations thereof.

9. The composition of claim 8 comprising one or more quaternary ammonium surfactants selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, and tricetylmonium chloride.

10. The composition of claim 8 comprising one or more amidoamine surfactants selected from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamidopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, and stearamidoethyldiethylamine.

11. The composition of claim 1, wherein the one or more fatty compounds include at least one ester group.

12. The composition of claim 11, wherein the one or more fatty compounds are selected from dibutyl adipate, propylene glycol dicaprylate/dicaprate, butylene glycol dicaprylate/dicaprate, glycol distearate, glycol stearate, ethylhexyl hydroxystearate, isopropyl isostearate, isopropyl myristate, isopropyl palmitate, isopropyl stearate, methyl glucose sesquistearate, methyl laurate, methyl stearate, oleyl oleate, propylene glycol monolaurate, propylene glycol monopalmitostearate, triacetin, sucrose distearate, ppg-20 methyl glucose ether distearate, diethyl sebacate, diisopropyl adipate, ethyl oleate, diisopropyl dilinoleate, coco-caprylate/caprate, and methyl salicylate.

13. The composition of claim 1, wherein the one or more fatty compounds are diesters.

14. The composition of claim 13, wherein the diesters are selected from diethyl succinate, dibutyl succinate, diethylhexyl succinate, diisopropyl sebacate, diethyl sebacate, dibutyl sebacate, diethylhexyl malate, diethylhexyl maleate, dicapryl adipate, dicaprylyl maleate, neopentyl glycol dicaprate, propylene glycol dicaprylate/dicaprate, neopentyl glycol diisostearate, neopentyl glycol diethylhexanoate, neopentyl glycol diheptanoate, dicaprylyl carbonate, or combinations thereof.

15. The composition of claim 1, wherein the one or more fatty compounds are selected from propylene glycol dicaprylate/dicaprate, propylene glycol dicaprylate, dicaprylyl carbonate, dicaprylyl ether, or combinations thereof.

16. The composition of claim 1, wherein the composition forms a lamellar structure upon application to a wet or damp surface.

17. A cosmetic composition comprising:
(a) about 5 to about 40 wt. % of one or more alpha hydroxy acid esters selected from diethyl tartrate, ethyl lactate, and triethyl citrate;
(b) about 2 to about 8 wt. % of one or more fatty alcohols;
(c) about 0.1 to about 5 wt. % of one or more cationic surfactants; and
(d) about 50 to about 80 wt. % of propylene glycol dicaprylate/dicaprate; and
(e) optionally, about 0.01 to about 5 wt. % of one or more miscellaneous ingredients;
provided the composition is translucent or transparent,
the composition is essentially free from water, mono-alcohols having from 2 to 6 carbon atoms, and polyols having from 2 to 8 carbon atoms,
the composition forms a lamellar structure upon application to a wet or damp surface, and
all percentages by weight are based on a total weight of the composition.

18. A cosmetic composition consisting of:
(a) about 5 to about 99 wt. % of one or more alpha hydroxy acid esters;
(b) about 0.1 to about 10 wt. % of one or more fatty alcohols;
(c) about 0.1 to about 10 wt. % of one or more cationic surfactants;
(d) one or more fatty compounds;
(e) optionally, about 0.01 to about 5 wt. % of one or more miscellaneous ingredients;
provided the composition is translucent or transparent,
the composition is essentially free from water, mono-alcohols having from 2 to 6 carbon atoms, and polyols having from 2 to 8 carbon atoms,
the composition forms a lamellar structure upon application to a wet or damp surface, and
all percentages by weight are based on a total weight of the composition.

19. A method for treating hair comprising applying the cosmetic composition of claim 1 to the hair.

20. The method of claim 19, wherein the composition is applied to wet or damp hair.
